(19) United States
(12) Patent Application Publication
(10) Pub. No.: US 2022/0227720 A1
(43) Pub. Date: Jul. 21, 2022

(54) DHODH INHIBITORS AND THEIR USE AS ANTIVIRAL AGENTS

(71) Applicants: UNIVERSITAT HAMBURG, Hamburg (DE); AIX-MARSEILLE UNIVERSITE, Marseille (FR)

(72) Inventors: Chris Meier, Jork (DE); Matthias Winkler, Hamburg (DE); Katharina Pfaff, Hamburg (DE); Nora Constanze Fohrmann, Hamburg (DE); Gilles Querat, Cabries (FR); Xavier de Lamballerie, Ensues la Redonne (FR)

(21) Appl. No.: 17/609,135

(22) PCT Filed: May 6, 2020
(86) PCT No.: PCT/EP2020/062629

§ 371 Date: Nov. 5, 2021

(30) Foreign Application Priority Data
May 7, 2019 (EP) 19173183.5
May 7, 2019 (LU) 101207

(51) Int. Cl. C07D 265/22; C07C 235/24; C07C 321/28; C07C 233/54; A61P 31/12; C07C 271/28; C07C 275/42; C07D 209/08; C07D 213/79; C07D 317/46; C07D 235/08; C07D 239/96; C07C 311/08

(52) U.S. Cl. CPC C07D 265/22; C07C 235/24; C07C 321/28; C07C 233/54; A61P 31/12; C07C 271/28; C07C 311/08; C07D 209/08; C07D 213/79; C07D 317/46; C07D 235/08; C07D 239/96; C07C 275/42

(57) ABSTRACT

The present invention relates to a compound, or a dimer or a pharmaceutically acceptable salt or solvate of said compound or dimer, for use in a method for the treatment of a disease, disorder or condition caused by an RNA virus, said compound having the general structure shown in Formula (I).

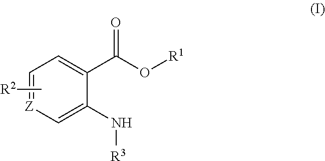

The present invention relates to anthranilic acid compounds useful as DHODH inhibitors, pharmaceutical compositions containing the compounds, and the use of the compounds and compositions in methods of treatment of a disease, disorder or condition caused by an RNA virus.

While the demand for nucleotides is covered by the salvage pathway in resting cells, fast proliferating cells, like cells of the immune system, tumor cells, or virally infected cells are highly dependent on the de novo nucleotide synthesis. Accordingly, the uses of inhibitors of the de novo nucleotide biosynthetic pathways have been investigated in the past.

Dihydroorotate dehydrogenase (DHODH) is a mitochondrial enzyme involved in pyrimidine metabolism. DHODH catalyzes the fourth step of the de novo synthesis of uridine monophosphate (UMP), which is afterwards converted to all other pyrimidine nucleotides. An inhibition of DHODH and thus a suppression of de novo synthesis of UMP leads to a downregulation of the intracellular number of pyrimidine nucleotides. This can lead to limitations in cell growth and proliferation.

Munier-Lehmann et al. (J. Med. Chem. 2013, 56, 3148-3167) report about the potential use of DHODH inhibitors in several therapeutic fields, such as the treatment of autoimmune diseases, e.g. rheumatoid arthritis or multiple sclerosis, the prophylaxis of transplant rejection, the treatment of cancer, e.g. leukemia or malignant melanoma, the treatment of viral and parasite infections, e.g. malaria, as well as in crop science.

One of the most potent DHODH inhibitors is brequinar with an IC₅₀ of about 10 nM. It has been investigated for cancer treatment and as an immunosuppressive drug in clinical trials. However, due to a narrow therapeutic window and inconsistent pharmacokinetics the further development was cancelled.

Another well described DHODH inhibitor is teriflunomide that derives from the prodrug leflunomide. It is the first approved DHODH inhibitor for the treatment of rheumatoid arthritis and is also used against multiple sclerosis. Disadvantages of this drug are a long plasma half-life and liver toxicity.

Chen et al. (Transplant Immunology 23 (2010) 180-184) describe the use of two DHODH inhibitor compounds, ABR-222417 and ABR-224050, as immunosuppressive agent. In particular, ABR-222417 is reported to result in a marked increase in graft survival time when screened in a low-responder heart allograft transplantation model in rats.

WO 2005/075410 discloses the use of certain DHODH inhibitor compounds for the treatment of autoimmune diseases, inflammatory diseases, organ transplant rejection and malignant neoplasia.

Compared to autoimmune diseases and oncology, virology is a rather novel field of application of DHODH inhibitors. US 2014/0080768 A1 and WO 2009/153043 A1 generally mention the potential use of DHODH inhibitors for various diseases and disorders including some viral diseases, besides autoimmune diseases, immune and inflammatory diseases, cancer, destructive bone disorders and others.

As regards DNA-viruses, Marschall et al. (Antiviral Res. 2013, 100, 640-648) focused on the in vivo antiviral efficacy of 3-(2,3,5,6-tetrafluoro-3′-trifluoromethoxy-biphenyl-4-ylcarbomyl)thiophene-2-carboxylic acid by inhibition of cytomegalovirus replication in mice. With respect to RNA-viruses, Wang et al. (J. Virol. 85 (2011), 6548-6556) investigated the inhibition of dengue virus (DENV) by NITD-982. While this compound demonstrated some in vitro potency, it did not show any efficacy in a mouse model.

Until now no DHODH inhibitor is used in antiviral therapy.

Therefore, there is still a need for compounds overcoming the above-mentioned drawbacks and problems. Accordingly, it is an object of the present invention to provide compounds which are suitable as antiviral agents and especially suitable to treat diseases, disorders or conditions caused by RNA viruses. In particular, it would be desirable to provide compounds having a high antiviral efficacy and at the same time a broad therapeutic window, i.e. a broad range of doses at which the therapeutic benefit is achieved without resulting in unacceptable side-effects or toxicity. Moreover, it would be desirable that the compounds are characterized by acceptable pharmacokinetic parameters, like ADME (Absorption, Distribution, Metabolism, and Excretion) properties. In addition, the compounds should be obtainable easily and in high yields avoiding cumbersome routes of synthesis.

It has surprisingly been found that the above problems are solved by the compounds defined below and in the appended claims.

Accordingly, in a first aspect the present invention relates to a compound, or a dimer or a pharmaceutically acceptable salt or solvate of said compound or dimer, for use in a method for the treatment of a disease, disorder or condition caused by an RNA virus, said compound having the general structure shown in Formula I:

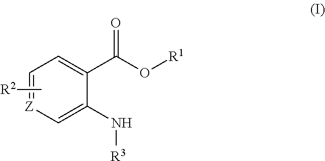

wherein:

Z is C or N, and preferably is C;

R¹ is H, alkyl, cycloalkyl, heterocyclyl, —C(O)-alkyl or a pharmaceutically acceptable cation, wherein the alkyl or —C(O)-alkyl can be unsubstituted or optionally substituted with one or more moieties which can be the same or different, each moiety being independently selected from the group consisting of —OC(O)-alkyl, —OC(O)O-alkyl, heterocyclyl, aryl and heteroaryl,

or optionally the —C(O)—O—R¹-group is joined to the —NH—R³-group to form together with the aromatic ring shown in Formula (I) a hetero ring system;

R² is one or more substituents independently selected from the group consisting of H, alkyl, alkenyl, alkynyl, aryl, alkoxy, aryloxy, halogen, haloalkyl, hydroxyalkyl and —NO₂, wherein each of said alkyl, alkenyl, alkynyl, aryl, alkoxy and aryloxy can be unsubstituted or optionally substituted with one or more moieties which can be the same or different, each moiety being independently selected from the group consisting of hydroxyl, halogen, alkyl, haloalkyl, aryl, haloaryl, alkylaryl, arylalkyl, cycloalkyl, aryloxy, alkoxy substituted with aryl, alkyl substituted with heterocyclyl, aryl substituted with haloaklkyl, aryl substituted with cycloalkyl, aryl substituted with arylalkyl, aryl substituted with alkoxy, aryl substituted with aryloxy, aryl substituted with —O-arylalkyl, aryl substituted with aryl, aryloxy, aryloxy substituted with alkyl, aryloxy substituted with cycloalkyl and aryloxy substituted with arylalkyl,

or optionally R² represents two substituents which are joined to form together with the aromatic ring shown in Formula (I) a substituted or unsubstituted ring or hetero ring system, or optionally at least one of R² is joined to the —NH—R³-group to form together with the aromatic ring shown in Formula (I) a hetero ring system;

R³ is —C(O)-alkyl, —C(O)O-alkyl, —C(O)NH-alkyl, —C(O)— cycloalkyl, —C(O)O-cycloalkyl, —C(O)NH-cycloalkyl, —C(O)-aryl, —C(O)O-aryl, —C(O) NH-aryl, —C(O)-heteroaryl, —C(O)O— heteroaryl, —C(O)NH-heteroaryl, aryl substituted with R⁵, heteroaryl substituted with R⁵, —S(O₂)—R⁹ or

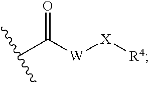

wherein

W is —(CH₂)ₙ—, —O—(CH₂)ₙ—, —NH—(CH₂)ₙ—, —(CH₂)ₚ-L-(CH₂)_{q}—, —O—(CH₂)ₚ-L-(CH₂)_{q}— or —NH— (CH₂)ₚ-L-(CH₂)_{q}—,

n is an integer from 1 to 6;

p and q are integers independently selected from 0 to 6;

L is a linking group selected from the group consisting of heteroaryl, aryl, heterocyclyl and cycloalkyl;

X is O, S, NH, CH₂, S(O) or C(O), and preferably is O, S or NH;

R⁴ is aryl or heteroaryl, wherein said aryl or heteroaryl can be unsubstituted or optionally substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl;

R⁵ is aryloxy or arylalkyl or optionally R⁵ represents two substituents linked to each other to form together with the aryl or heteroaryl a polycyclic ring system, wherein preferably the polycyclic ring system is a naphthalene or fluorene; and

R⁹ is alkyl, cycloalkyl or —W—X—R⁴.

Preferably, R³ is —C(O)-alkyl, —C(O)O-alkyl, —C(O)NH-alkyl, —C(O)-cycloalkyl, —C(O)O-cycloalkyl, —C(O)NH-cycloalkyl, —C(O)-aryl, —C(O)O-aryl, —C(O)NH-aryl, —C(O)-heteroaryl, —C(O)O— heteroaryl, —C(O)NH-heteroaryl, aryl substituted with R⁵, heteroaryl substituted with R⁵, or

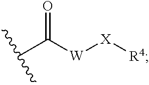

wherein

W is —(CH₂)ₙ—, —O—(CH₂)ₙ—, —NH—(CH₂)ₙ—, —(CH₂)ₚ-L-(CH₂)_{q}—, —O—(CH₂)ₚ-L-(CH₂)_{q}— or —NH—(CH₂)ₚ-L-(CH₂)_{q}—,

n is an integer from 1 to 6;

p and q are integers independently selected from 0 to 6;

L is a linking group selected from the group consisting of heteroaryl, aryl, heterocyclyl and cycloalkyl;

X is O, S, NH, or CH₂, and preferably is O, S or NH;

R⁴ is aryl or heteroaryl, wherein said aryl or heteroaryl can be unsubstituted or optionally substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl and haloalkylaryl; and

R⁵ is aryloxy or arylalkyl or optionally R⁵ represents two substituents linked to each other to form together with the aryl or heteroaryl a polycyclic ring system, wherein preferably the polycyclic ring system is a naphthalene or fluorene.

It has surprisingly been found that the above-defined compounds have a high antiviral efficacy. In particular, the compounds have been found to have a high antiviral activity against a wide spectrum of viruses, especially a wide spectrum of RNA viruses. Hence, the compounds have been found to be promising candidates for in vivo applications as broad-spectrum antiviral drug.

The above-defined compounds have been found to act by inhibition of a host enzyme. This brings several advantages compared to inhibitors targeting viral enzymes, such as (i) a higher barrier to resistance, (ii) a broad coverage of different viruses that goes along with a preventative preparation for new emerging virus infections, and (iii) allowing antiviral therapy of virus infections without druggable viral targets. In particular, the above-defined compounds have been found to act by inhibition of the host enzyme DHODH. The DHODH sets itself apart from other cellular targets by the fact that the host cell is able to overcome its temporary inhibition by the steady supply of pyrimidine nucleotides for resting cells via salvage pathway.

In a preferred embodiment, the compound to be used according to the invention is represented by formula (I) and Z is C. Accordingly, the aromatic ring shown in Formula I is preferably a phenyl ring and the compounds to be used according to the invention are anthranilic acid derivatives.

In a preferred embodiment, the compound to be used according to the invention is represented by formula (I) and R¹ is H. Thus, the compound is preferably present as a free anthranilic acid. However, under physiological conditions the free acid is deprotonated resulting in a negatively charged carboxylate. In order to mask this negative charge under physiological conditions, prodrug moieties can be installed at the R¹ position to enable penetration through the cell membrane and allow the release of the active carboxylic acid inside the cell.

Accordingly, besides H, R¹ can be a pharmaceutically acceptable cation or alkyl, cycloalkyl, heterocyclyl or —C(O)-alkyl, wherein the alkyl or —C(O)-alkyl can be unsubstituted or optionally substituted with one or more moieties which can be the same or different, each moiety being independently selected from the group consisting of —OC(O)-alkyl, —OC(O)O-alkyl, heterocyclyl, aryl and heteroaryl. For example, R¹ can be —C(O)-alkyl to form an anhydride prodrug moiety. Moreover, R¹ can be alkyl substituted with —OC(O)O-alkyl to form an alkoxycarbonyloxyalkyl (POC) moiety. Furthermore, R¹ can be alkyl substituted with aryl which in turn is substituted with an —O—C(O)-alkyl group to form an acyloxybenzyl moiety.

In one embodiment, R¹ is selected from the group consisting of H,

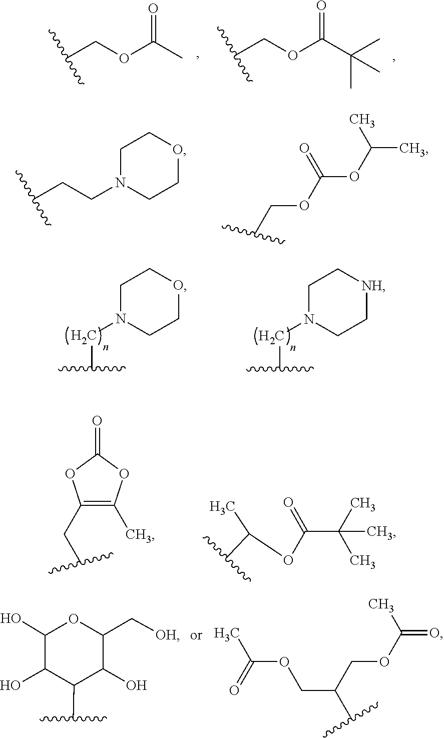

and preferably is H.

In a further embodiment, R¹ is selected from the group consisting of H,

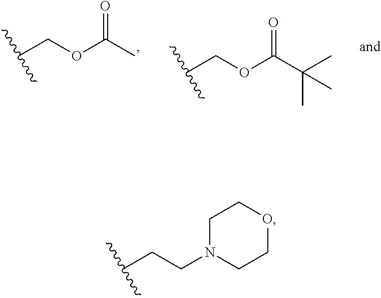

and preferably is H.

In a further embodiment, the —C(O)—O—R¹-group can be joined to the —NH—R³-group to form together with the aromatic ring shown in Formula (I) a hetero ring system. Preferably, this hetero ring system can comprise an oxazine or quinazolinone. Accordingly, the hetero ring system formed by joining the —C(O)—O—R¹-group with the —NH—R³-group together with the aromatic ring shown in Formula (I) can preferably be represented by

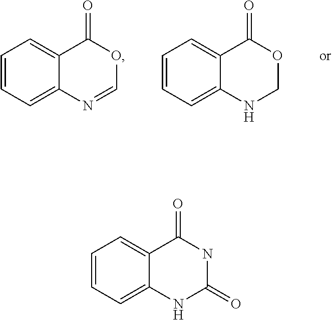

Accordingly, in this embodiment the compounds to be used according to the invention can preferably be represented by

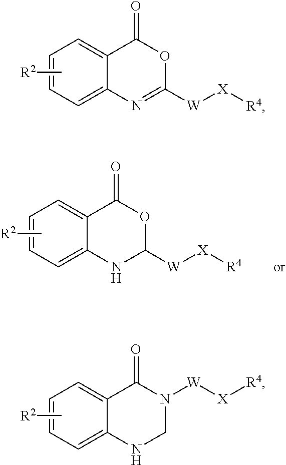

with W preferably being —(CH₂)ₙ—.

Furthermore, the compounds to be used according to the invention are characterized in that the aromatic ring of the anthranilic core is substituted by one or more R² substituents. In a one embodiment, R² is a substituent, in particular one substituent, as defined above.

In a preferred embodiment, R² is aryl, preferably phenyl, which can be unsubstituted or optionally substituted with one or more moieties which can be the same or different, each moiety being independently selected from the group consisting of alkyl, aryl, halogen, haloaryl, alkylaryl, arylalkyl, cycloalkyl, aryloxy, alkoxy substituted with aryl, alkyl substituted with heterocyclyl.

In a more preferred embodiment, R² is

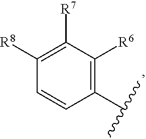

wherein

R⁶ is H; halogen, preferably F; or aryl, preferably phenyl;

R⁷ is H; halogen, preferably F; alkyl, preferably methyl; or aryl, preferably phenyl; and

R⁸ is H; cycloalkyl, preferably cyclohexyl; aryl, preferably phenyl; haloaryl, preferably 4-F-phenyl; arylalkyl, preferably 4-ethyl-phenyl, 4-pentyl-phenyl; alkylaryl, preferably benzyl; aryloxy, preferably phenyloxy; arylalkoxy, preferably benzyloxy; or heterocyclylalkyl, preferably morpholinomethyl.

In an even more preferred embodiment, R² is

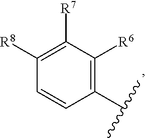

wherein R⁶, R⁷ and R⁸ are selected as shown in the following table:

| R⁶ | R⁷ | R⁸ |
|---|---|---|
| H | H | Ph |
| Ph | H | H |
| H | Ph | H |
| H | F | Ph |
| H | H | 4-F-Ph |
| F | H | 4-Et-Ph |
| H | H | 4-Pentyl-Ph |
| H | H | 4-Benzyl |
| H | H | Cyclohexyl |
| H | H | OPh |
| H | CH₃ | OCH₂Ph |
| H | F | OCH₂Ph |
| H | H | Morpholinomethyl |

In another preferred embodiment, R² is alkynyl, preferably ethynyl, which can be unsubstituted or optionally substituted with a moiety selected from the group consisting of aryl, aryl substituted with alkyl, aryl substituted with haloaklkyl, aryl substituted with cycloalkyl, aryl substituted with arylalkyl, aryl substituted with alkoxy, aryl substituted with aryloxy, aryl substituted with —O-arylalkyl, aryl substituted with aryl, aryloxy, aryloxy substituted with alkyl, aryloxy substituted with cycloalkyl and aryloxy substituted with arylalkyl.

More preferably, R² is alkynyl, preferably ethynyl, which is substituted with a moiety selected from the group consisting of phenyl; phenyl substituted with 4-haloalkyl like 4-CF₃; phenyl substituted with 4-alkyl like 4-C₄H₉ or 4-C₆H₁₃; phenyl substituted with 4-alkoxy like 4-ethoxy or 4-pentoxy; phenyl substituted with 4-aryloxy like 4-phenyloxy; phenyl substituted with arylalkoxy like 4-benzyloxy; phenyl substituted with 4-aryl like 4-phenyl; phenyl substituted with 4-cycloalkyl like 4-cyclohexyl; phenyl substituted with 4-arylalkyl like 4-benzyl; and alkyl, preferably methyl or butyl, which is substituted with aryloxy, preferably phenyloxy, which in turn is substituted with 2-alkyl, like 2-sec-butyl, 2-cycloalkyl, like 2-cyclohexyl, or 2-arylalkyl, like 2-benzyl.

In an even more preferred embodiment, R² is alkynyl, preferably ethynyl, which is substituted with a moiety selected from the group consisting of

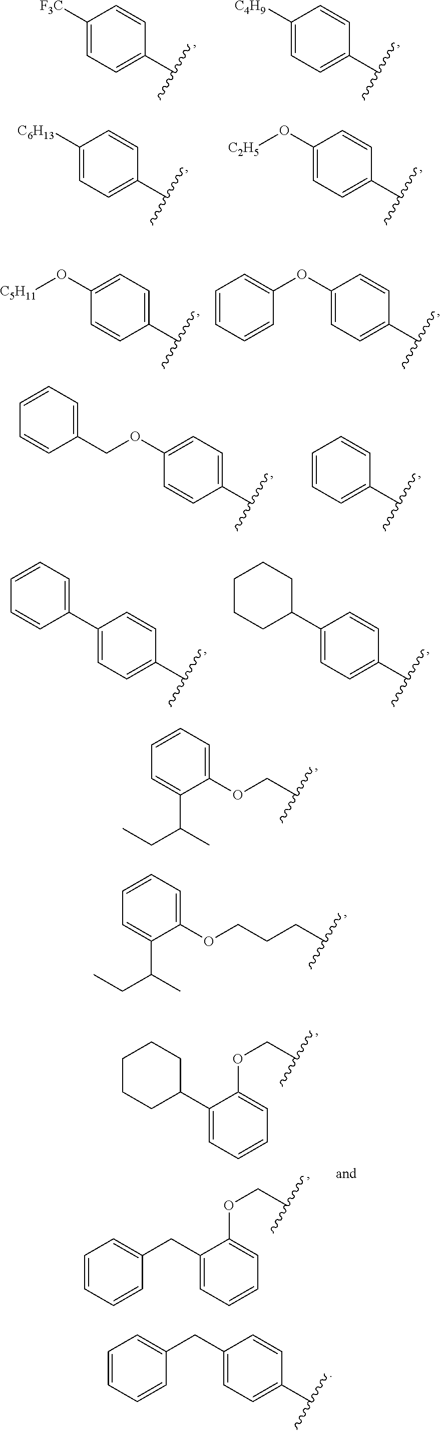

In another preferred embodiment, R² is H; alkyl, preferably 5-alkyl like 5-methyl or 5-tBu; halogen, wherein halogen is preferably selected from F, Cl and Br, and/or wherein halogen is preferably 5-halogen like 5-Br or 5-F or 4-halogen like 4-F; alkoxy, preferably 5-alkoxy like 5-methoxy or 4-alkoxy like 4-methoxy; haloalkyl, preferably 5-CF₃; NO₂, preferably 5-NO₂; or aryl, preferably phenyl or biphenyl like 5-phenyl or 5-biphenyl.

In another embodiment, R² represents two substituents which are joined to form together with the aromatic ring shown in Formula (I) a ring system or a hetero ring system. The ring system and the hetero ring system can be unsubstituted or optionally substituted, e.g. by a ring system substituent as defined herein. In particular, the ring system can comprise an aryl, such as e.g. a phenyl, annelated to the aromatic ring of Formula (I). In this embodiment the phenyl ring of the anthranilic core of the compounds of Formula (I) and the phenyl ring annelated to it form a polycylic aromatic hydrocarbon, such as e.g. a naphthalene. In a further embodiment, the hetero ring system formed by joining two R² substituents together with the aromatic ring shown in Formula (I) is

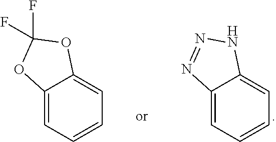 [SMILES: FC1(F)Oc2ccccc2O1 ; c1ccc2[nH]nnc2c1]

Accordingly, in this embodiment the compounds to be used according to the invention can be represented by

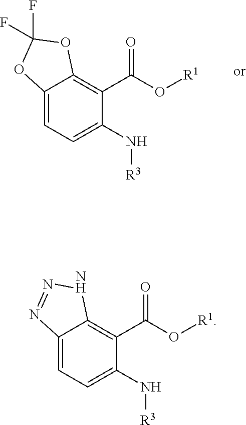

In a further embodiment, at least one of R² is joined to the —NH—R³-group to form together with the aromatic ring shown in Formula (I) a hetero ring system. In particular, this hetero ring system can comprise a benzimidazole moiety, such as

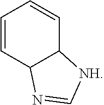

Accordingly, in this embodiment the compounds to be used according to the invention can be represented by

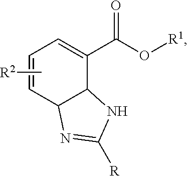

such as

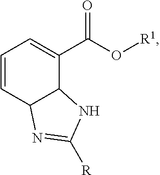

with R being e.g. alkyl.

Furthermore, it is preferred that R³ is selected from the group consisting of —C(O)-alkyl, —C(O)—O-alkyl, —C(O)—NH-alkyl, aryl and

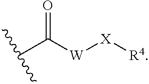

In a preferred embodiment, R³ is —C(O)-alkyl or —C(O)—O-alkyl or —C(O)—NH-alkyl, and preferably —C(O)-alkyl. In this embodiment, it is particularly preferred that the alkyl is methyl, ethyl or isopropyl. Most preferably, the alkyl is ethyl.

In another preferred embodiment, R³ is aryl, preferably phenyl, wherein the aryl, or preferably phenyl, is substituted with R⁵, with R⁵ being arylalkyl, preferably benzyl, or aryloxy, preferably phenoxy.

In another preferred embodiment, R³ is

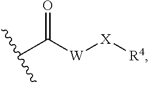

with W preferably being —(CH₂)ₙ—, —O—(CH₂)ₙ— or —NH—(CH₂)ₙ—, and more preferably being —(CH₂)ₙ—, and n being 1 to 6, preferably 1 to 3, more preferably 1;

X being O or S or SO or CO, and preferably O or S, and more preferably O; and

R⁴ preferably being phenyl substituted with 2-sec-butyl, 4-sec-butyl, 2-tert-amyl, 2-tert-butyl, 4-tert-butyl, 2-tert-butyl-4-methyl, 2,6-di-tert-butyl-4-methyl, 2-methyl, 2,6-dimethyl, 3,5-dimethyl, 2,4-dimethyl, 2,3,5-trimethyl, 2,4,6-trimethyl, 2-isopropyl, 2-methyl-5-isopropyl, 5-methyl-2-isopropyl, 2,6-di-isopropyl, 2-ethyl, 2-propyl, 2-ethoxy, 2-cyclohexyl, 2-cyclopentyl, 2-adamantanyl-4-methyl, 2-benzyl, 2-benzyl-4-chloro, 2-phenyl, 3-phenyl, 4-phenyl, 1-naphthyl, 2-naphthyl, 4-phenoxy, 2,6-dichloro, 2-iodo or 2-bromo-4-methyl.

In another embodiment, R³ is

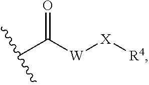

with W preferably being —(CH₂)ₚ-L-(CH₂)_{q}—, —O—(CH₂)ₚ-L-(CH₂)_{q}— or —NH—(CH₂)ₚ-L-(CH₂)_{q}—, with the linking group L preferably being heteroaryl and more preferably 1,4-triazole or 1,5-triazole, and p and q being 0 to 6, preferably 0 to 4, and more preferably 1 to 4, such as 1 to 2. X and R⁴ are preferably defined as listed above. Examples of such compounds are shown in e.g. Schemes 3 and 13.

In another embodiment, R³ is preferably —C(O)-heteroaryl, wherein the heteroaryl can be unsubstituted or substituted and preferably is N-pyrrole, N-indole or N-carbazole, or —C(O)—NH-aryl, wherein the aryl can be unsubstituted or substituted, wherein the aryl preferably is unsubstituted and/or wherein the aryl preferably is phenyl.

In another embodiment, R³ is preferably —S(O₂)—R⁹, with R⁹ being, alkyl, cycloalkyl or —W—X—R⁴, wherein W, X and R⁴ are as defined above. Preferably, R⁹ is alkyl or cycloalkyl, and more preferably alkyl, like e.g. methyl, ethyl, n-propyl, n-butyl, iso-propyl or iso-butyl.

In another embodiment, when R⁵ is aryloxy or arylalkyl, the aryl moiety of these groups is preferably substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl. Accordingly, the compounds according to the invention can for instance be illustrated by the following formula:

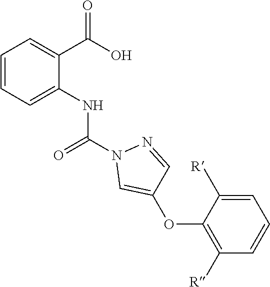

in which R′ and R″ can independently be selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl: For example, R′ and R″ can independently be selected from e.g. —CF₃, tert. butyl and morpholinyl.

In another embodiment, when R³ is —C(O)NH-aryl, the aryl moiety is preferably substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl. Accordingly, the compounds according to the invention can for instance be illustrated by the following formula:

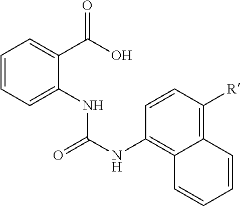

in which R′ can be —X—R″, with X being C, CO, NH or S and R″ can be halogen, alkyl, cycloalkyl, heterocyclyl, aryl, heteroaryl, alkoxy, aryloxy, arylalkyl, alkylaryl, haloaryl, haloalkylaryl, haloalkyl or trialkylsilyl.

Redoxal, a known DHODH inhibitor, is a compound synthesized via the coupling of two anthranilic acid compounds. Hence, also the compounds to be used according to the present invention can be present in the form of a dimer, i.e. two molecules having the structure of formula (I) can be coupled to form a dimer. In particular, the two molecules are coupled via substituents R² and/or R³. Thus, the phenyl ring of the first anthranilic acid core is coupled via R² to R² or R³ of the phenyl ring of the second anthranilic acid core. Alternatively, the phenyl ring of the first anthranilic acid core is coupled via R³ to R² or R³ of the phenyl ring of the second anthranilic acid core. Suitable routes of synthesis to provide dimers according to the invention are described below.

As used above, and throughout this disclosure, the following terms, unless otherwise indicated, shall be understood to have the following meanings:

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkenyl chain. Non-limiting examples of suitable alkenyl groups include ethenyl and propenyl. The term “substituted alkenyl” means that the alkenyl group may be substituted by one or more substituents which may be the same or different, each substituent being independently selected from the group consisting of alkyl, aryl and cycloalkyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable arylalkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined herein. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, re-places an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously re-places two available hydrogens on two adjacent carbon atoms (one H on each carbon) on a ring system. Examples of such moiety are methylene dioxy, ethylenedioxy, —C(CH₃)₂— and the like which form moieties such as, for example:

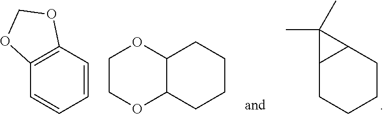

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

It should also be noted that any heteroatom with unsatisfied valences in the text, schemes, examples and Tables herein is assumed to have the hydrogen atom(s) to satisfy the valences.

When any variable (e.g., aryl, etc.) occurs more than one time in any constituent or in Formula I, its definition on each occurrence is independent of its definition at every other occurrence.

Prodrugs and solvates of the compounds of the invention are also contemplated herein. The term “prodrug”, as employed herein, denotes a compound that is a drug precursor which, up-on administration to a subject, undergoes chemical conversion by metabolic or chemical processes to yield a compound of Formula I or a salt and/or solvate thereof.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts can also be useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydro-chlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of Formula I, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds of the invention may, for example, be substantially free of other isomers, or may be ad-mixed, for example, as racemates or with all other, or other selected, stereoisomers.

The compounds of Formula I have pharmacological properties. In particular, the compounds of Formula I have found to be inhibitors of DHODH. Moreover, it has been found that the compounds of Formula I have a very high antiviral efficacy against various RNA viruses in an up to one-digit nanomolar range. In a preferred embodiment, the compounds to be used according to the invention are characterized by an IC₅₀ of less than 0.7 μM, preferably less than 0.3 μM, more preferably less than 0.1 μM, like less than 0.08 μM or less than 0.05 μM or less than 0.03 μM, wherein the IC₅₀ values are generated in an assay using TOSV, HAZV, TAHV, RVFV, YFV, DENV, TBEV, ZIKV or VEEV using the assay described in the examples herein. Moreover, the compounds are characterized by a low cytotoxicity, such as a CC₅₀ of more than 8 μM, preferably more than 10 μM, more preferably more than 15 μM, like more than 30 μM or more than 50 μM or more than 70 μM or more than 100 μM. Hence, the compounds of Formula I are expected to be useful in the therapy of viral diseases, in particular in the treatment of treatment of a disease, disorder or condition caused by an RNA virus.

As used herein, the term “RNA virus” refers to a virus that has RNA (ribonucleic acid) as its genetic material. This nucleic acid is usually single-stranded RNA (ssRNA) but may be double-stranded RNA (dsRNA). In particular, the RNA virus is a virus that belongs to Group III, Group IV or Group V of the Baltimore classification system of classifying viruses.

Preferably, the RNA virus which can be affected by the compounds of Formula I is selected from the group consisting of bunya viruses including Toscana virus (TOSV), hazara virus (HAZV), tahyna virus (TAHV), rift valley fever virus (RVFV), Lassa virus (LSAV), Punta Toro phlebovirus (PTV) and Crimean-Congo hemorrhagic fever orthonairovirus (CCHFV); flavi viruses including yellow fever virus (YFV), dengue virus (DENV), tick-borne encephalitis virus (TBEV), zika virus (ZIKV) and Hepatitis C virus (HCV); toga viruses including venezuelan equine encephalitis virus (VEEV), Sindbis virus (SINV) and Chikungunya virus (CHIKV); mononegaviruses including Ebola virus (EBOV), Marburg virus (MARV), Human parainfluenza virus 3 (HPIV-3), Nipah virus (NiV) and Vesicular stomatitis virus (VSV); picorna viruses including coxsackievirus (CV); nidoviruses including Severe acute respiratory syndrome-related coronavirus (SARS-CoV), Severe acute respiratory syndrome-related coronavirus 2 (SARS-CoV-2) and Middle-East respiratory syndrome-related coronavirus (MERS-CoV); and reoviruses including reovirus type 1 (Reo-1).

Hence, also disclosed is a method of treating a mammal (e.g., human) having a disease or condition associated with a virus, in particular RNA virus, by administering a therapeutically effective amount of at least one compound of Formula I, or a pharmaceutically acceptable salt or solvate of said compound to the mammal.

Moreover, pharmaceutical compositions comprising a compound of Formula I are also disclosed herein. The pharmaceutical composition comprises at least one compound of Formula I, or a pharmaceutically acceptable salt or solvate of said compound, and at least one pharmaceutically acceptable carrier. For pre-paring pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration. Aerosol preparations suitable for inhalation may include solutions and solids in powder form, which may be in combination with a pharmaceutically acceptable carrier, such as an inert compressed gas, e.g. nitrogen. Also included are solid form preparations that are intended to be converted, shortly before use, to liquid form preparations for either oral or parenteral administration. Such liquid forms include solutions, suspensions and emulsions. Preferably the compound to be used according to the invention is administered orally.

In a second aspect, the present invention relates to a compound, or a dimer or a pharmaceutically acceptable salt or solvate of said compound or dimer, having the general structure shown in Formula I:

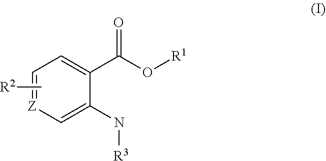

wherein:

Z is C or N, and preferably is C;

R¹ is H, alkyl, cycloalkyl, heterocyclyl, —C(O)-alkyl or a pharmaceutically acceptable cation, wherein the alkyl or —C(O)-alkyl can be unsubstituted or optionally substituted with one or more moieties which can be the same or different, each moiety being independently selected from the group consisting of —OC(O)-alkyl, —OC(O)O-alkyl, heterocyclyl, aryl and heteroaryl,

or optionally the —C(O)—O—R¹-group is joined to the —NH—R³-group to form together with the aromatic ring shown in Formula (I) a hetero ring system;

R² is one or more substituents independently selected from the group consisting of H, alkyl, alkenyl, alkynyl, aryl, alkoxy, aryloxy, halogen, haloalkyl, hydroxyalkyl and —NO₂, wherein each of said alkyl, alkenyl, alkynyl, aryl, alkoxy and aryloxy can be unsubstituted or optionally substituted with one or more moieties which can be the same or different, each moiety being independently selected from the group consisting of hydroxyl, halogen, alkyl, haloalkyl, aryl, haloaryl, alkylaryl, arylalkyl, cycloalkyl, aryloxy, alkoxy substituted with aryl, alkyl substituted with heterocyclyl, aryl substituted with haloaklkyl, aryl substituted with cycloalkyl, aryl substituted with arylalkyl, aryl substituted with alkoxy, aryl substituted with aryloxy, aryl substituted with —O-arylalkyl, aryl substituted with aryl, aryloxy, aryloxy substituted with alkyl, aryloxy substituted with cycloalkyl and aryloxy substituted with arylalkyl,

or optionally R² represents two substituents which are joined to form together with the aromatic ring shown in Formula I a substituted or unsubstituted ring or hetero ring system,

or optionally at least one of R² is joined to the —NH—R³-group to form together with the aromatic ring shown in Formula (I) a hetero ring system;

R³ is —C(O)-alkyl, —C(O)O-alkyl, —C(O)NH-alkyl, —C(O)— cycloalkyl, —C(O)O-cycloalkyl, —C(O)NH-cycloalkyl, —C(O)-aryl, —C(O) O-aryl, —C(O)NH-aryl, —C(O)-heteroaryl, —C(O)O— heteroaryl, —C(O)NH-heteroaryl, aryl substituted with R⁵, heteroaryl substituted with R⁵, —S(O₂)—R⁹, or

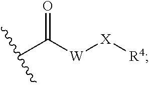

wherein

W is —(CH₂)ₙ—, —(CH₂)ₙ—, —NH—(CH₂)ₙ—, —(CH₂)ₚ-L-(CH₂)_{q}—, —O— (CH₂)ₚ-L-(CH₂)_{q}— or —NH—(CH₂)ₚ-L-(CH₂)_{q}—,

n is an integer from 1 to 6;

p and q are integers independently selected from 0 to 6;

L is a linking group selected from the group consisting of heteroaryl, aryl, heterocyclyl and cycloalkyl;

X is O, S, NH, CH₂, S(O) or C(O), and preferably is O, S or NH;

R⁴ is aryl or heteroaryl, wherein said aryl or heteroaryl can be unsubstituted or optionally substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl;

R⁵ is aryloxy or arylalkyl or optionally R⁵ represents two substituents linked to each other to form together with the aryl or heteroaryl a polycyclic ring system; and

R⁹ is alkyl, cycloalkyl or —W—X—R⁴;

with the proviso that, when R³ is —C(O)-alkyl and the alkyl is methyl, then R² is not H, halogen, phenyl, biphenyl or 2-Cl-4-CF₃-phenoxy;

with the further proviso that, when R³ is —C(O)-alkyl and the alkyl is ethyl or cyclopropyl, then R² is not alkyl substituted with a phenyl ring which is unsubstituted at the 4-position, alkenyl substituted with a phenyl ring which is unsubstituted at the 4-position, alkynyl substituted with a phenyl ring which is unsubstituted at the 4-position, alkyoxy substituted with a phenyl ring which is unsubstituted at the 4-position, or aryloxy wherein the aryl of the aryloxy is a phenyl ring which is unsubstituted at the 4-position;

with the further proviso that, when R³ is —C(O)-alkyl and the alkyl is substituted with halogen, then R² is not alkyl or halogen;

with the further proviso that, when R³ is aryl substituted with R⁵ and R⁵ is aryloxy, then the aryloxy is not phenoxy;

with the further proviso that, when R³ is —C(O)NH-aryl, then the aryl is not substituted with aryl or halogen.

Preferably, in the compounds of the second aspect R³ is —C(O)— alkyl, —C(O)O-alkyl, —C(O)NH-alkyl, —C(O)-cycloalkyl, —C(O)O— cycloalkyl, —C(O)NH-cycloalkyl, —C(O)-aryl, —C(O)O-aryl, —C(O) NH-aryl, —C(O)-heteroaryl, —C(O) O-heteroaryl, —C(O)NH— heteroaryl, aryl substituted with R⁵, heteroaryl substituted with R⁵, or

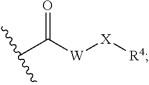

wherein

W is —(CH₂)ₙ—, —O—(CH₂)ₙ—, —NH—(CH₂)ₙ—, —(CH₂)ₚ-L-(CH₂)_{q}—, —O—(CH₂)ₚ-L-(CH₂)_{q}— or —NH—(CH₂)ₚ-L-(CH₂)_{q}—,

n is an integer from 1 to 6;

p and q are integers independently selected from 0 to 6;

L is a linking group selected from the group consisting of heteroaryl, aryl, heterocyclyl and cycloalkyl;

X is O, S, NH, CH₂, and preferably is O, S or NH;

R⁴ is aryl or heteroaryl, wherein said aryl or heteroaryl can be unsubstituted or optionally substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, and haloaryl, haloalkylaryl; and

R⁵ is aryloxy or arylalkyl or optionally R⁵ represents two substituents linked to each other to form together with the aryl or heteroaryl a polycyclic ring system;

with the proviso that, when R³ is —C(O)-alkyl and the alkyl is methyl, then R² is not H, halogen, phenyl, biphenyl or 2-Cl-4-CF₃-phenoxy;

with the further proviso that, when R³ is —C(O)-alkyl and the alkyl is ethyl or cyclopropyl, then R² is not alkyl substituted with a phenyl ring which is unsubstituted at the 4-position, alkenyl substituted with a phenyl ring which is unsubstituted at the 4-position, alkynyl substituted with a phenyl ring which is unsubstituted at the 4-position, alkyoxy substituted with a phenyl ring which is unsubstituted at the 4-position, or aryloxy wherein the aryl of the aryloxy is a phenyl ring which is unsubstituted at the 4-position;

with the further proviso that, when R³ is —C(O)-alkyl and the alkyl is substituted with halogen, then R² is not alkyl or halogen;

with the further proviso that, when R³ is aryl substituted with R⁵ and R⁵ is aryloxy, then the aryloxy is not phenoxy.

Preferred definitions of substituents Z and R¹ to R³ of the compounds of the invention are described above in connection with the first aspect of the invention.

In a particularly preferred embodiment, the compound according to the invention has one of the following structures:

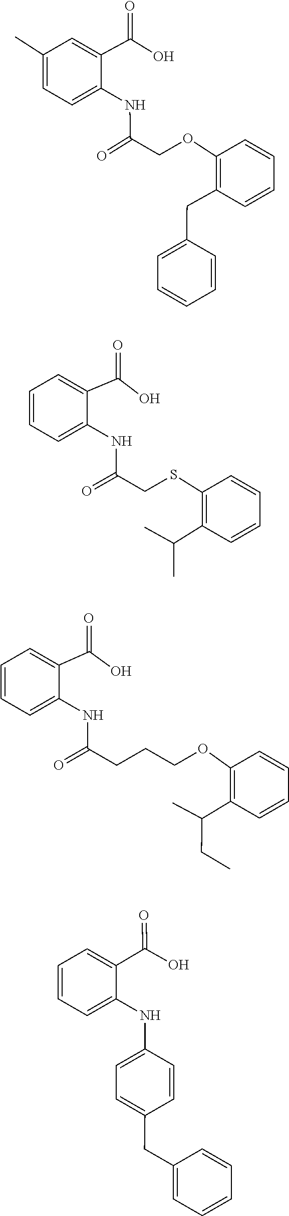 [SMILES: Cc1ccc(NC(=O)COc2ccccc2Cc2ccccc2)c(C(=O)O)c1 ; CC(C)c1ccccc1SCC(=O)Nc1ccccc1C(=O)O ; CCC(C)c1ccccc1OCCCC(=O)Nc1ccccc1C(=O)O ; O=C(O)c1ccccc1Nc1ccc(Cc2ccccc2)cc1]

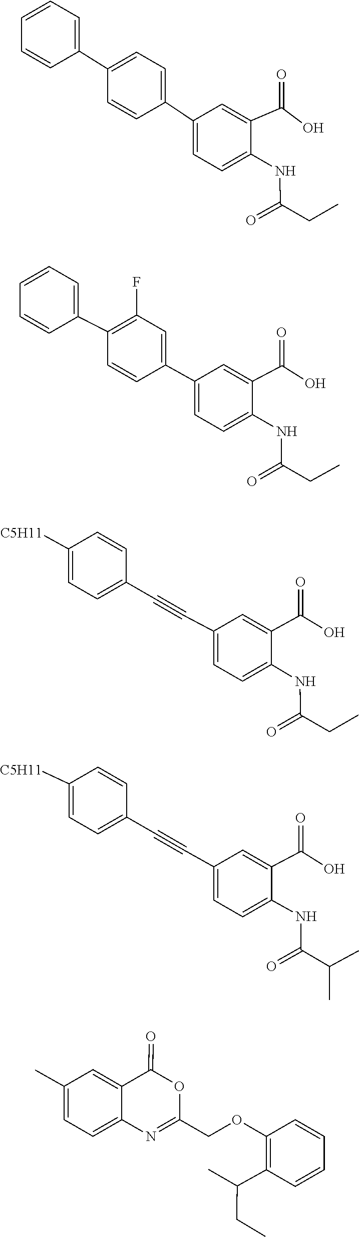

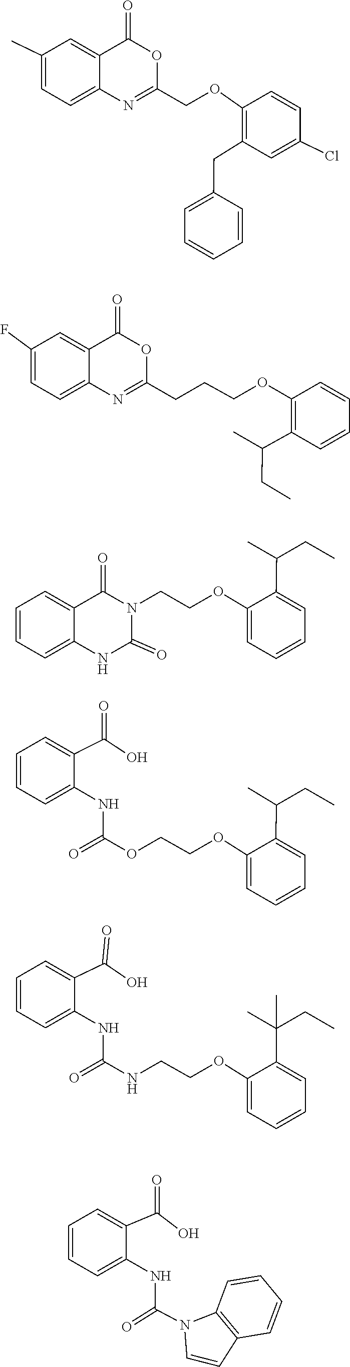

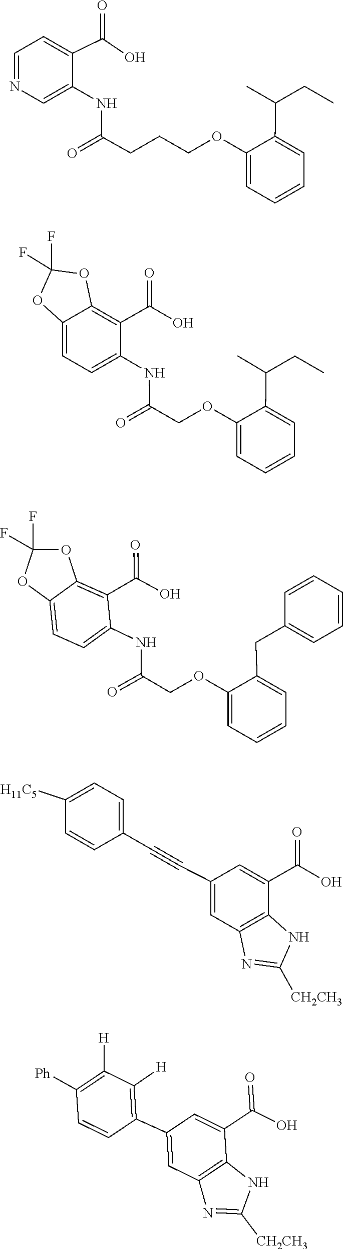

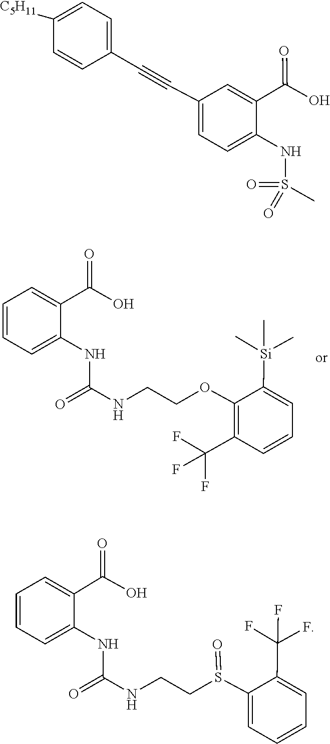

In a further particularly preferred embodiment, the compound according to the invention has one of the following structures:

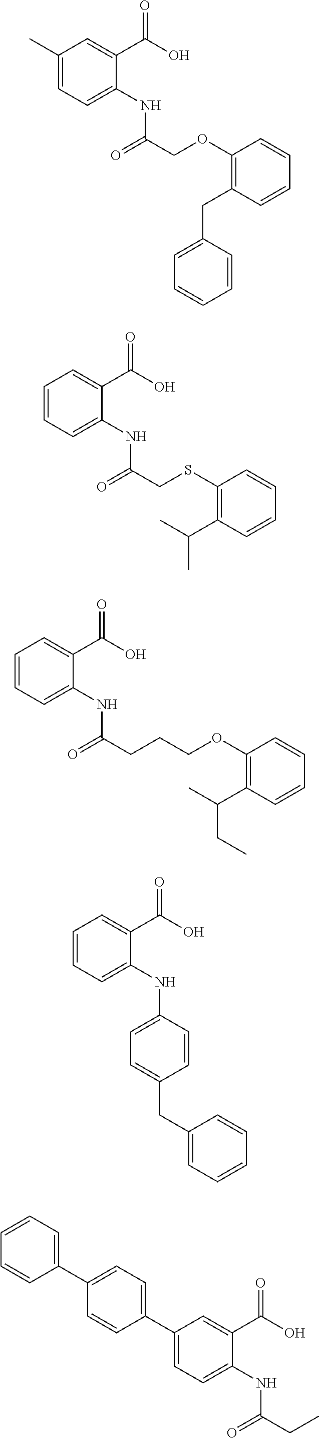 [SMILES: Cc1ccc(NC(=O)COc2ccccc2Cc2ccccc2)c(C(=O)O)c1 ; CC(C)c1ccccc1SCC(=O)Nc1ccccc1C(=O)O ; CCC(C)c1ccccc1OCCCC(=O)Nc1ccccc1C(=O)O ; O=C(O)c1ccccc1Nc1ccc(Cc2ccccc2)cc1 ; CCC(=O)Nc1ccc(-c2ccc(-c3ccccc3)cc2)cc1C(=O)O]

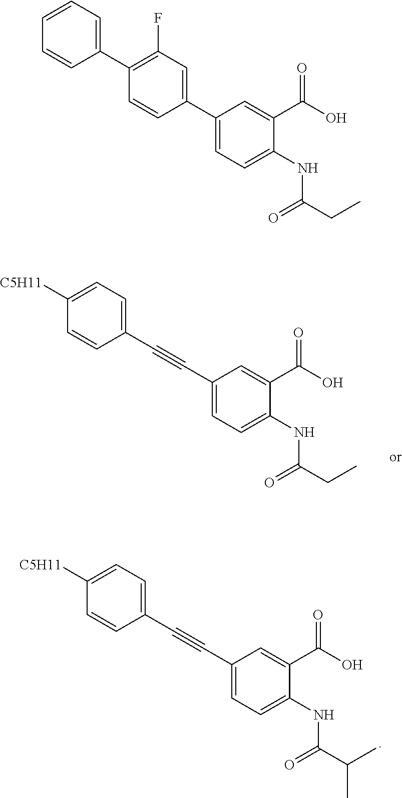

The compounds according to the second aspect of the invention are particularly suitable to be used in accordance with the first aspect of the invention, i.e. for the treatment of a disease, disorder or condition caused by an RNA virus. Moreover, the compounds according to the second aspect of the invention are suitable to be used for the treatment of autoimmune diseases, e.g. rheumatoid arthritis or multiple sclerosis, the prophylaxis of transplant rejection, the treatment of cancer, e.g. leukemia or malignant melanoma, the treatment of viral and parasite infections, e.g. malaria, as well as in crop science.

The compounds of formula (I) described above, i.e. the compounds to be used in accordance with the first aspect of the invention as well as the compounds according to the second aspect of the invention, can be prepared in high yields using short routes of synthesis.

The invention disclosed herein is exemplified by the following examples which should not be construed to limit the scope of the disclosure. Alternative mechanistic pathways and analogous structures will be apparent to those skilled in the art.

The following solvents and reagents may be referred to by their abbreviations:

rac-BINAP (2,2′-bis(diphenylphosphino)-1,1′-binaphthyl) racemate
B₂Pin₂ bis(pinacolato)diboron
DCC N,N′-dicyclohexylcarbodiimide
DMAP 4-dimethylaminopyridine
DME dimethoxyethane
DMF dimethylformamide
Et₃N or Net₃ triethylamine
Log D distribution coefficient
PBS phosphate buffered saline
PAMPA parallel artificial membrane permeability assay

### EXAMPLES

In general, the compounds of Formula I, wherein R³ is

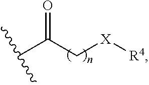

can be prepared through the general routes described below in Scheme 1.

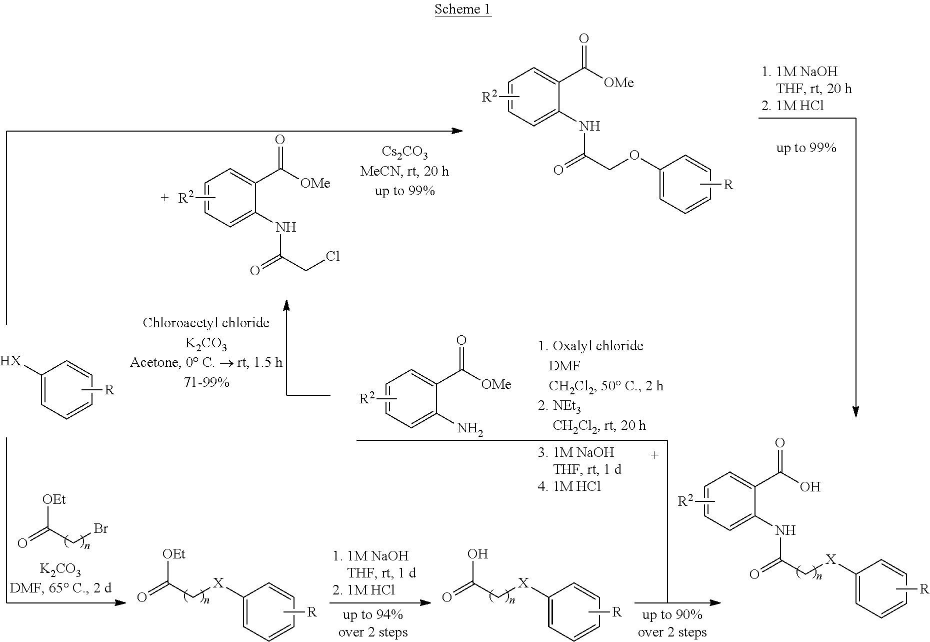

For example, R² can be H, 5-Me, 5-Br, 5-F, 5-OMe, 5-CF₃, 5-NO₂, 5-tBu, 4-F, 4-OMe, or annelated phenyl. X can be O, S, or NH. n can be 1 to 6. Moreover, R can be H or halogen or an aliphatic or aromatic moiety. For example, R can be selected from the group consisting of 2-sec-butyl, 4-sec-butyl, 2-tert-amyl, 2-tert-butyl, 4-tert-butyl, 2-tert-butyl-4-methyl, 2,6-di-tert-butyl-4-methyl, 2-methyl, 2,6-dimethyl, 3,5-dimethyl, 2,4-dimethyl, 2,3,5-trimethyl, 2,4,6-trimethyl, 2-isopropyl, 2-methyl-5-isopropyl, 5-methyl-2-isopropyl, 2,6-di-isopropyl, 2-ethyl, 2-propyl, 2-ethoxy, 2-cyclohexyl, 2-cyclopentyl, 2-adamantanyl-4-methyl, 2-benzyl, 2-benzyl-4-chloro, 2-phenyl, 3-phenyl, 4-phenyl, 1-naphthyl, 2-naphthyl, 4-phenoxy, 2,6-dichloro, 2-iodo, and 2-bromo-4-methyl.

Further derivatives of more lipophilic compounds of Formula I were synthesized via palladium catalyzed Suzuki cross coupling reaction according to Scheme 2.

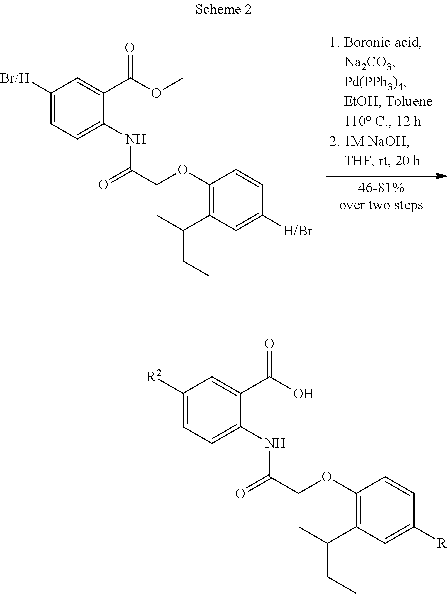

The structures were further modified by installing substituted phenyl residues at the 5 position of the anthranilic acid or in the 4′-position of the phenolic moiety. For example, R² can be Ph, 3,5-Cl-Ph, 3-CF₃-Ph, biphenyl, wherein R is ═H. Furthermore, R can be Br or 3-CF₃-Ph, wherein R² is H.

Anthranilic acids bearing a 1,4-triazole moiety in the linking unit between the anthranilic acid core structure and the phenol ether were synthesized using copper catalyzed click reaction according to Scheme 3.

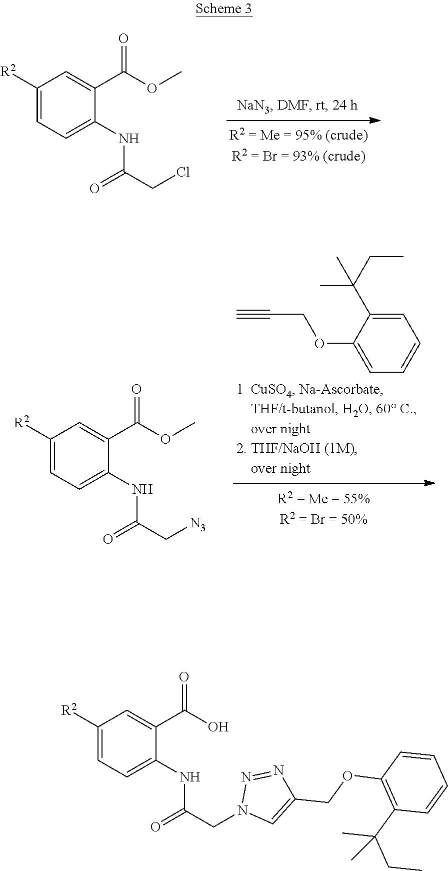

Compounds of Formula I, wherein R³ is aryl, were prepared through the general routes described below in Scheme 4.

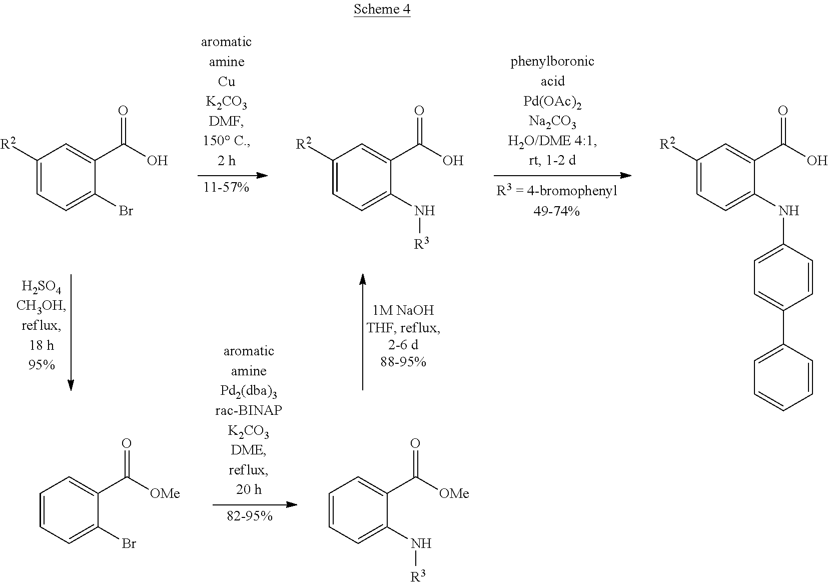

Unsubstituted and 5-OMe-substituted (R²) fenamic acid compounds with various aromatic residues (R³) were synthesized in that manner, such as R³=3-phenoxyphenyl, 4-phenoxyphenyl, 4-benzylphenyl, 2-fluorenyl, [1,1′-biphenyl]-3-yl, 4-bromophenyl and [1,1′-biphenyl]-4-yl via 4-bromophenyl.

In order to mask the negative charge of the carboxylate under physiological conditions, prodrug moieties can be installed at the R¹ position to enable penetration through the cell membrane and allow the release of the active carboxylic acid inside the cell. The synthesis was carried out directly starting from the synthesized anthranilic acids by treatment with the appropriate alkyl halides under basic conditions or by a nucleophilic opening of in situ formed 1,3-oxazine derivatives with the respective alcohol. The synthetic routes towards these prodrugs are outlined in Scheme 5.

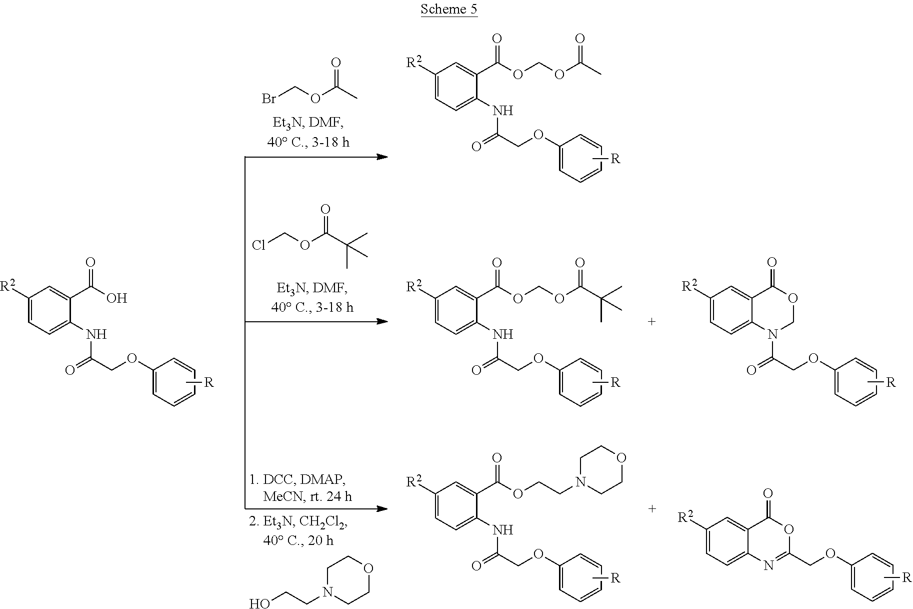

R² substituted anthranilic acids bearing different amides were synthesized using different acid chlorides followed by palladium catalyzed Suzuki cross coupling reaction according to Scheme 6.

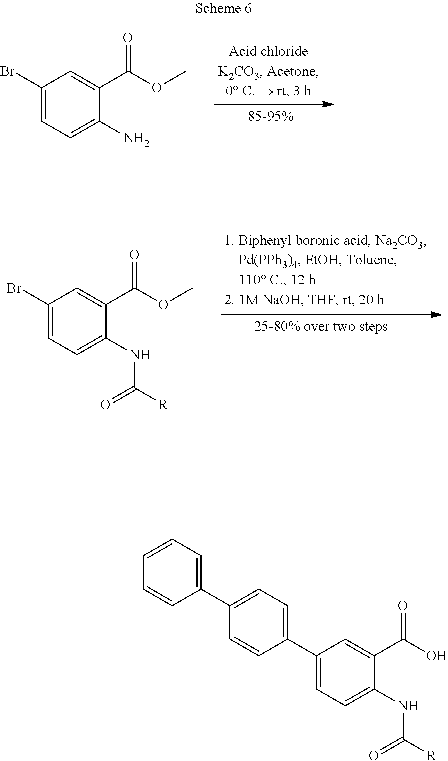

For instance, R can be methyl, ethyl, isopropyl, cyclopropyl, n-butyl.

Various compounds, wherein R² is phenyl or substituted phenyl, were synthesized according to Scheme 7.

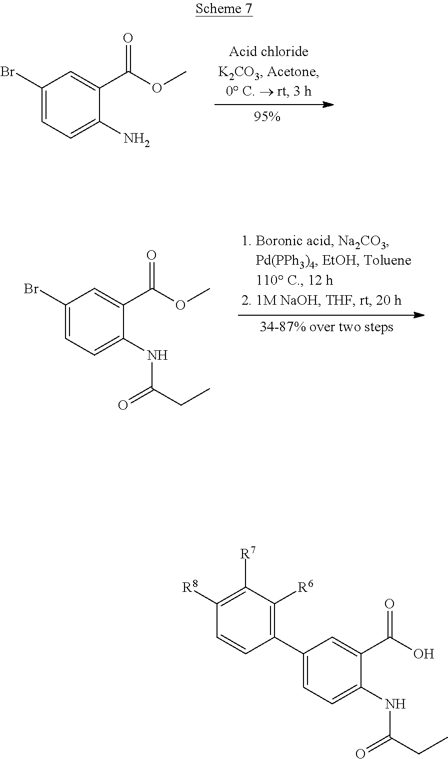

Suitable combinations of R⁶, R⁷ and R⁸ are given above in the general description.

4-Pentynylbenzene ethynyl anthranilic acids bearing different amides were synthesized using different acid chlorides followed by palladium and copper catalyzed Sonogashira cross coupling reaction according to Scheme 8.

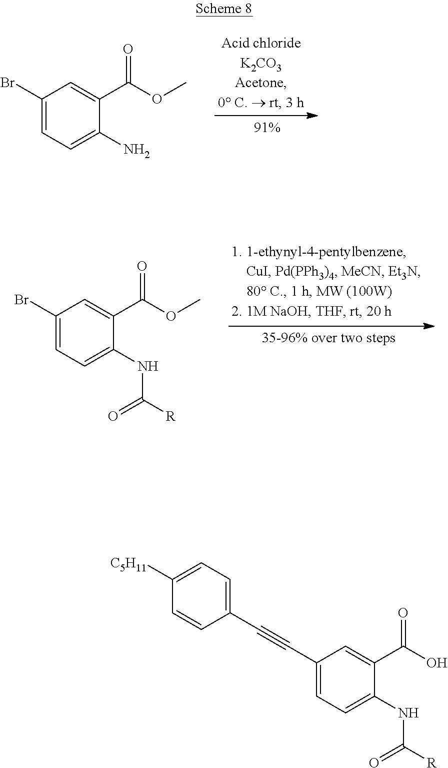

R was selected from methyl, ethyl, isopropyl, cyclopropyl, tert-butyl, and iso-valeryl.

Furthermore, various 5-ethynyl anthranilic acids were synthesized according to Scheme 9.

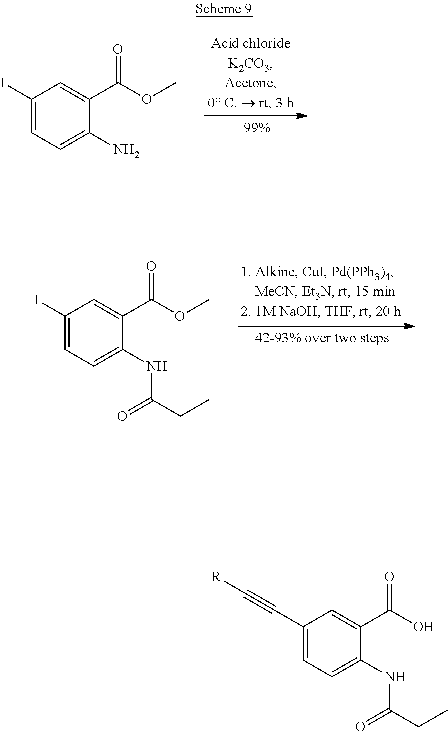

Examples of suitable substituents R are given above in the general description.

Dimers of the compounds of the invention were synthesized via Suzuki-Miyaura cross coupling according to Scheme 10. These compounds were synthesized in analogy to Redoxal, a known DHODH inhibitor.

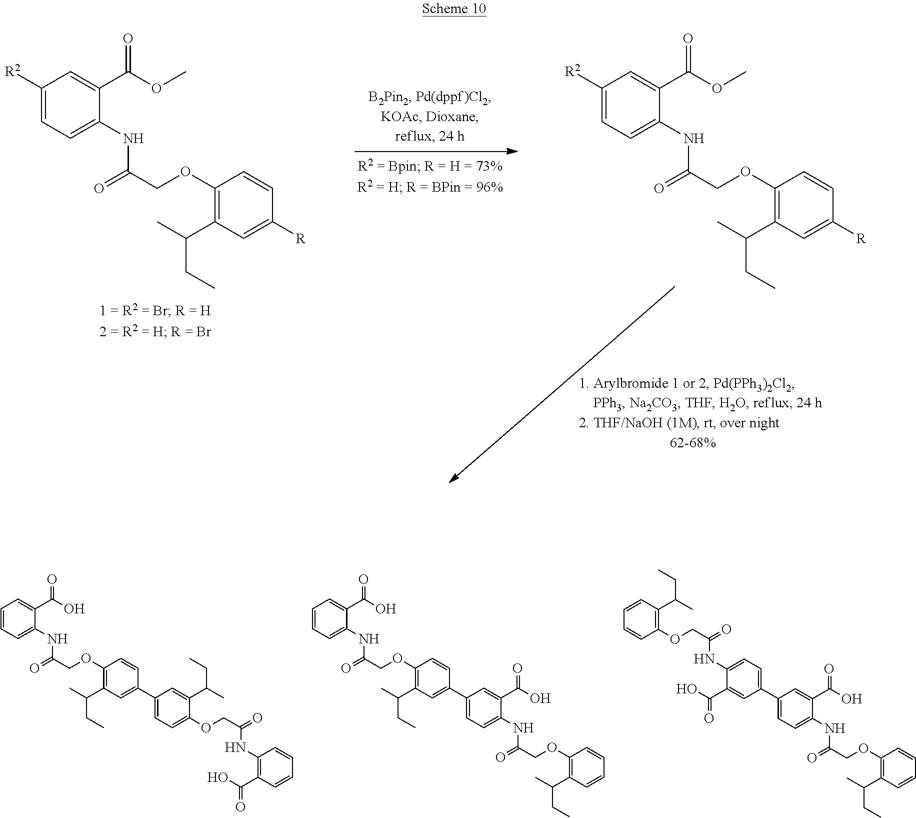

Compounds with R¹ being joined to the —NH—R³-group to form together with the aromatic ring of formula (I) a hetero ring system, such as a hetero ring system comprising an oxazine moiety, were synthesized according to Scheme 11.

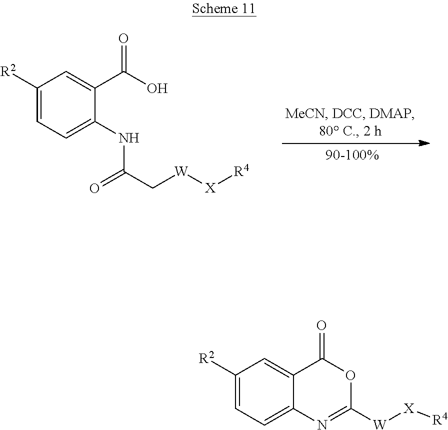

For example, R² can be H, methyl, Br, F, —CF₃, —OMe.

Compounds with R¹ being joined to the —NH—R³-group to form together with the aromatic ring of formula (I) a hetero ring system, such as a hetero ring system comprising a quinazoline moiety, were synthesized according to Scheme 12.

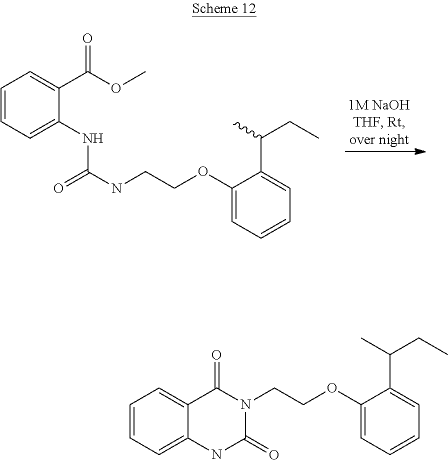 [SMILES: CCC(C)c1ccccc1OCCNC(=O)Nc1ccccc1C(=O)OC ; CCC(C)c1ccccc1OCCn1c(=O)[nH]c2ccccc2c1=O]

Anthranilic acids bearing a 1,5-triazole moiety in the linking unit between the anthranilic acid core structure and the phenol ether were synthesized according to Scheme 13.

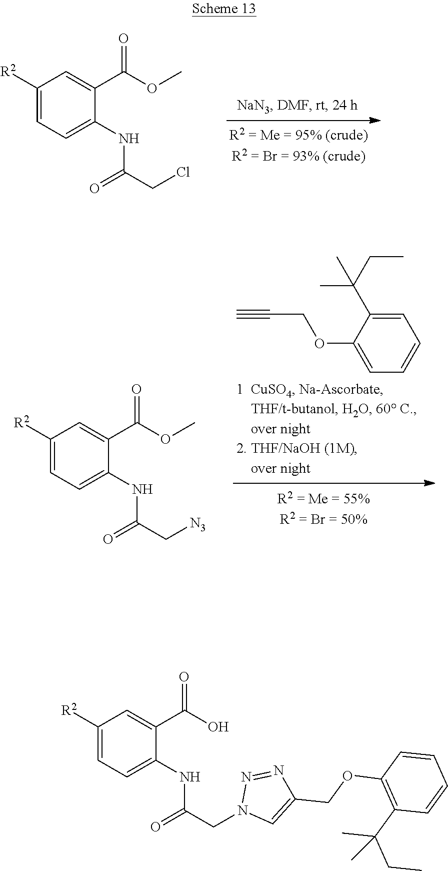

Compounds with one R² being linked to the —NH—R³-group were synthesized according to Scheme 14, wherein R can be ethyl, R′ is preferably —C₅H₁₁, R⁶ is H, R⁷ is H and R⁸ is phenyl:

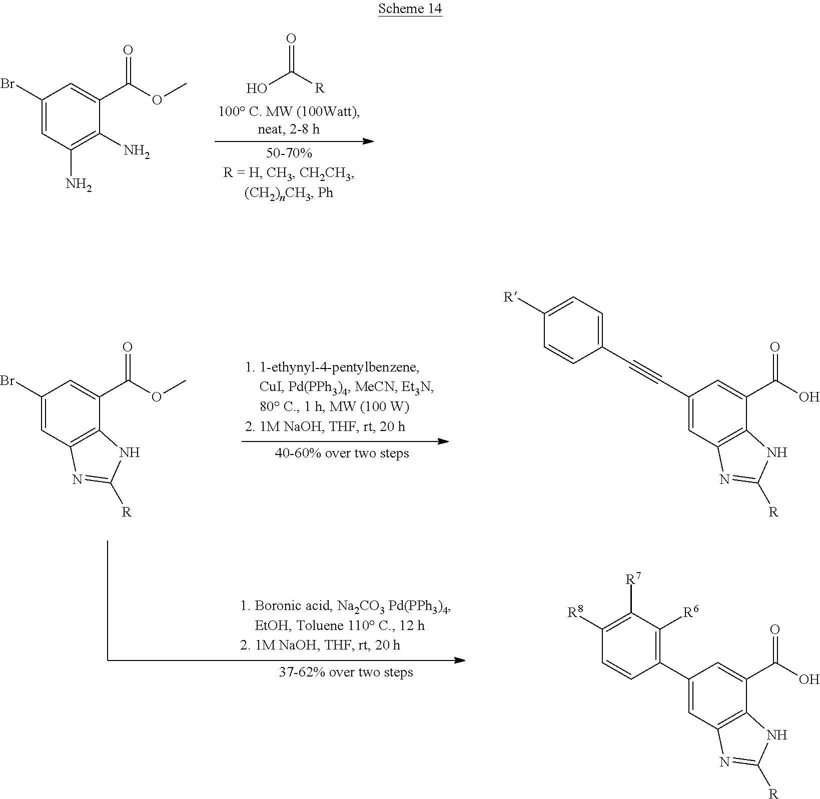

### Example 1

Preparation of

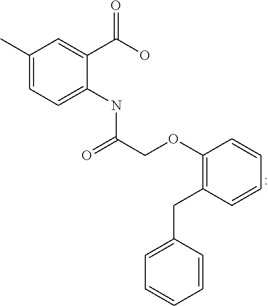 [SMILES: Cc1ccc(NC(=O)COc2ccccc2Cc2ccccc2)c(C(=O)O)c1]

4.45 g (26.9 mmol) methyl-2-amino-5-methylbenzoate and 18.6 g (135 mmol) potassium carbonate were suspended in 200 mL acetone. 5.36 mL (67.4 mmol) 2-chloracetylchloride were added dropwise and the reaction mixture was stirred at room temperature for 2 hours. After addition of ethyl acetate and a saturated aqueous solution of sodium bicarbonate the phases were separated. The organic phase was washed with demineralised water and brine, dried over sodium sulfate, filtrated and the solvent was evaporated. The product precipitated by addition of petroleum ether 50-70 and was filtrated.

Yield: 6.08 g (21.1 mmol, 93%) of colorless crystals (methyl-2-(2-chloroacetamido)-5-methyl-benzoate).

To a suspension of 4.09 g (22.2 mmol) 2-benzylphenol and 14.5 g (44.4 mmol) caesium carbonate in 120 mL acetonitrile 4.88 g (20.2 mmol) methyl-2-(2-chloroacetamido)-5-methyl-benzoate were added. The reaction mixture was stirred for 17 hours at room temperature. Subsequently the solvent was evaporated and the crude product was purified by column chromatography (di-chloromethane/petroleum ether 50-70). The obtained methyl ester was dissolved in 100 mL THE and 40 mL of an aqueous sodium hydroxide solution (1 M) were added. After stirring 15 hours at room temperature the reaction mixture was adjusted to pH 1 with hydrochloric acid (1 M) and diluted with dichloromethane. After phase separation the aqueous phase was extracted with dichloromethane three times. The combined organic layers were dried over sodium sulfate, filtrated and the solvent was evaporated. The crude product was purified by crystallization from dichloromethane/petroleum ether 50-70.

Yield over two steps: 7.14 g (19.0 mmol, 94%) of colorless crystals.

R_{f}(CH₂Cl₂/CH₃OH 19:1 v/v): 0.50.

¹H-NMR: δ [ppm] (500 MHz, DMSO-d₆): 13.62 (s, 1H, COOH), 11.85 (s, 1H, CONH), 8.58 (d, ³J_{H,H}=8.5 Hz, 1H, H-3), 7.84 (d, ⁴J_{H,H}=1.9 Hz, 1H, H-6), 7.45 (dd, ³J_{H,H}=8.6 Hz, ⁴J_{H,H}=2.2 Hz, 1H, H-4), 7.26-7.22 (m, 4H, H-2″, H-3″), 7.22-7.10 (m, 3H, H-3′, H-5′, H-4″), 7.03 (dd, ³J_{H,H}=7.0 Hz, 1H, H-6′), 6.94 (m, 1H, H-4′), 4.69 (s, 1H, OCH₂), 4.18 (s, 2H, benzyl-CH₂), 2.31 (s, 3H, CH₃).

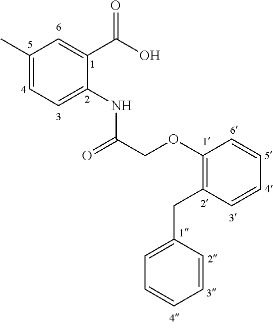 [SMILES: Cc1ccc(NC(=O)COc2ccccc2Cc2ccccc2)c(C(=O)O)c1]

¹³C-NMR: δ [ppm] (126 MHz, DMSO-d₆): =169.3 (COOH), 166.9 (CONH), 154.8 (C-1′), 140.8 (C-1″), 137.8 (C-2), 134.7 (C-4), 132.3 (C-5), 131.3 (C-6), 130.3 (C-3′), 129.9 (C-2′), 128.8 (2×C-2″/C-3″), 128.2 (2×C-2″/C-3″), 127.5 (C-5′), 125.7 (C4″), 121.6 (C-4′), 116.3 (C-1), 112.3 (C-6′), 67.8 (OCH₂), 34.9 (benzyl-CH₂), 20.2 (CH₃).

IR: \tilde{ν} [cm⁻¹]: 3599, 3228, 3023, 1665, 1587, 1518, 1490, 1450, 1432, 1301, 1271, 1226, 1108, 1067, 1018, 861, 798, 749, 699, 666, 617, 531.

HRMS (ESI⁺) m/z=calcd for C₂₃H₂₂NO₄: 376.1543 [M+H]⁺, found: 376.1550.

### Example 2

Preparation of

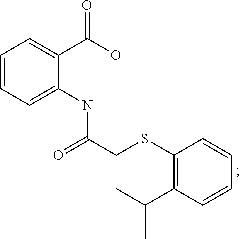

1.1 mL (1.1 g, 7.0 mmol) 2-Isopropylbenzenethiol, 1.94 g (14.0 mmol) potassium carbonate and 1.2 mL (2.2 g, 11 mmol) ethyl iodoacetate were suspended in 20 mL DMF. The reaction was stirred at 100° C. for 4 d and then diluted with water and di-chloromethane. After separation of the phases the aqueous phase was extracted twice with dichloromethane. The combined organic layers were washed with brine, dried over sodium sulfate, filtrated and the solvent was evaporated. To a solution of 1.67 g (7.00 mmol) ethyl 2-(2-isopropylphenylthio)acetate in 28 mL THE 21 mL of an aqueous sodium hydroxide solution (1 M) was added. The reaction was stirred at room temperature for 18 h, and then adjusted to pH 1 with hydrochloric acid (1 M). The reaction was diluted with dichloromethane and the phases were separated. After extraction of the aqueous phase with di-chloromethane twice the combined organic layers were dried over sodium sulfate, filtrated and the solvent was evaporated. The crude product was purified by column chromatography (di-chloromethane/methanol).

Yield: 1.01 g (4.80 mmol, 69%) of a colorless solid.

205 mg (0.975 mmol) 2-(2-Isopropylphenylthio)acetic acid, 261 mg (1.27 mmol) dicyclohexylcarbodiimide and 157 mg (1.36 mmol) N-hydroxysuccinimide in 30 mL acetonitrile were stirred at room temperature for 24 h. The suspension was filtrated and the solvent was evaporated. The crude product was purified by column chromatography (dichloromethane). 98 mg (0.32 mmol) N-succinimide 2-(2-isopropylphenylthio)acetate and 37 mg (0.27 mmol) anthranilic acid were dissolved in 5 mL acetonitrile and 0.05 mL (0.04 g, 0.3 mmol) diisopropylethylamine were added. The reaction was stirred at room temperature for 18 h and then the solvent was evaporated. The crude product was purified by column chromatography (dichloromethane/methanol) and by crystallization from dichloromethane/petroleum ether 50-70.

Yield: 69 mg (0.21 mmol, 79%) of a colorless solid.

R_{f} (CH₂Cl₂/CH₃OH 19:1 v/v): 0.37.

MP: 135° C.

¹H-NMR: δ [ppm] (400 MHz, DMSO-d₆): 13.61 (brs, 1H, COOH), 11.75 (s, 1H, NH), 8.51 (dd, ³J_{H,H}=8.5 Hz, ⁴J_{H,H}=0.9 Hz, 1H, H-3), 7.96 (dd, ³J_{H,H}=7.8 Hz, ⁴J_{H,H}=1.5 Hz, 1H, H-6), 7.59-7.53 (m, 1H, H-4), 7.35-7.32 (m, 1H, H-3′), 7.28 (dd, ³J_{H,H}=7.5 Hz, ⁴J_{H,H}=1.8 Hz, 1H, H-6′), 7.21-7.11 (m, 3H, H-5, H-4′, H-5′), 3.95 (s, 2H, CH₂), 3.45 (sept, ³J_{H,H}=6.8 Hz, 1H, CH), 1.17 (d, ³J_{H,H}=6.8 Hz, 6H, CH(CH₃)₂).

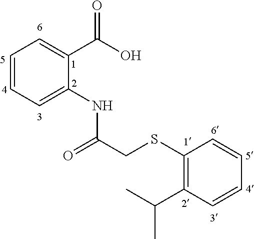 [SMILES: CC(C)c1ccccc1SCC(=O)Nc1ccccc1C(=O)O]

¹³C-NMR: δ [ppm] (101 MHz, DMSO-d₆) 169.2 (COOH), 167.5 (CONH), 147.2 (C-2′), 140.4 (C-2), 134.0 (C-4), 132.8 (C-1′), 131.1 (C-6), 128.0 (C-3′), 126.7, 126.6 (C-4′, C-5′), 125.5 (C-6′), 122.9 (C-5), 120.0 (C-3), 116.5 (C-1), 38.4 (CH₂), 29.7 (CH), 23.1 (CH(CH₃)₂).

IR: \tilde{ν} [cm⁻¹]: 2960, 2925, 2867, 1684, 1605, 1586, 1523, 1471, 1450, 1401, 1298, 1258, 1229, 1163, 1145, 1083, 1051, 1017, 796, 755, 699, 648.

HRMS (ESI⁺) m/z=calcd for C₁₈H₂₀NO₃S: 330.1159 [M+H]⁺, found: 330.1168.

### Example 3

Preparation of

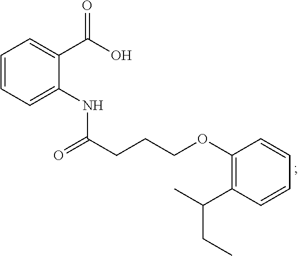 [SMILES: CCC(C)c1ccccc1OCCCC(=O)Nc1ccccc1C(=O)O]

0.50 mL (0.45 g, 3.0 mmol) (rac)-2-sec-butylphenol, 0.46 g (3.4 mmol) potassium carbonate and 0.57 mL (0.77 g, 3.3 mmol) ethyl 4-bromobutyrate were suspended in 5 mL DMF. The reaction was stirred at 65° C. for 2 d and then diluted with water and dichloromethane. After separation of the phases the aqueous phase was extracted twice with dichloromethane. The combined organic layers were washed with brine, dried over sodium sulfate, filtrated and the solvent was evaporated. The crude product was purified by column chromatography (petroleum ether 50-70/dichloromethane). Yield: 751 mg (2.84 mmol, 95%) of a yellow oil (ethyl 4-(rac)-(2-sec-butylphenoxy)butyrate).

To a solution of 111 mg (0.420 mmol) ethyl 4-(rac)-(2-sec-butylphenoxy)butyrate in 1.8 mL THE 1.3 mL of an aqueous sodium hydroxide solution was added. The reaction was stirred at room temperature for 16 h, and then adjusted to pH 1 with hydrochloric acid (1 M). The reaction was diluted with dichloro-methane and the phases were separated. After extraction of the aqueous phase with dichloromethane twice the combined organic layers were dried over sodium sulfate, filtrated and the solvent was evaporated. The crude product was purified by crystallization from dichloromethane/petroleum ether 50-70. Yield: 95 mg (0.40 mmol, 96%) of colorless crystals (4-(rac)-(2-sec-butylphenoxy)butyric acid).

To a solution of 190 mg (0.804 mmol) 4-(rac)-(2-sec-butylphenoxy)butyric acid in 5 mL dichloromethane 0.10 mL (0.15 g, 1.2 mmol) oxalyl chloride and a catalytic amount of DMF were added at 0° C. The reaction was heated at reflux for 3 h. After cooling to room temperature the solvent was evaporated. The obtained acid chloride was dissolved in dichloro-methane and added dropwise to a solution of 0.10 mL (0.12 g, 0.80 mmol) methyl anthranilate and 0.12 mL (0.088 g, 0.87 mmol) triethylamine in 5 mL dichloromethane. The reaction was stirred at room temperature for 2 h and then a saturated aqueous solution of sodium bicarbonate and dichloromethane were added to the mixture. After separation of the phases the aqueous phase was extracted with dichlormethane twice, the combined organic layers were dried over sodium sulfate, filtrated and the solvent was evaporated. The crude product was purified by column chromatography (petroleum ether 50-70/dichloromethane). Yield: 292 mg (0.790 mmol, 98%) of a colorless sirup (methyl 2-(4-(rac)-(2-sec-butyl-phenoxy)butanamido)benzoate).

To a solution of 243 mg (0.658 mmol) methyl 2-(4-(rac)-(2-sec-butylphenoxy)butanamido)benzoate in 2.6 mL THE 2.0 mL of an aqueous sodium hydroxide solution was added. The reaction was stirred at room temperature for 18 h, and then adjusted to pH 1 with hydrochloric acid (1 M). The reaction was diluted with dichloromethane and the phases were separated. After extraction of the aqueous phase with dichloromethane twice the combined organic layers were dried over sodium sulfate, filtrated and the solvent was evaporated. The crude product was purified by crystallization from dichloromethane/petroleum ether 50-70. Yield: 171 mg (0.482 mmol, 73%) of colorless crystals (title compound of Example 1).

R_{f} (CH₂Cl₂/CH₃OH 9:1 v/v): 0.25.

MP: 133° C.

¹H-NMR: δ [ppm] (400 MHz, DMSO-d₆): 13.60 (brs, 1H, COOH), 11.27 (s, 1H, NH), 8.53 (dd, ³J_{H,H}=8.3 Hz, ⁴J_{H,H}=0.6 Hz, 1H, H-3), 7.98 (dd, ³J_{H,H}=7.9 Hz, ⁴J_{H,H}=1.6 Hz, 1H, H-6), 7.61-7.55 (m, 1H, H-4), 7.16-7.08 (m, 3H, H-5, H-3′, H-5′), 6.92 (dd, ³J_{H,H}=8.8 Hz, 4J_{H,H}=1.0 Hz, 1H, H-6′), 6.90-6.84 (m, 1H, H-4′), 4.02 (t, ³J_{H,H}=6.1 Hz, 2H, H-9), 3.02 (sext, ³J_{H,H}=7.0 Hz, 1H, CH), 2.60 (t, ³J_{H,H}=7.5 Hz, 2H, H-7), 2.09 (quin, ³J_{H,H}=6.6 Hz, 2H, H-8), 1.60-1.40 (m, 2H, CH₂), 1.09 (d, ³J_{H,H}=7.0 Hz, 3H, CHCH₃), 0.73 (t, ³J_{H,H}=7.4 Hz, 3H, CH₂CH₃).

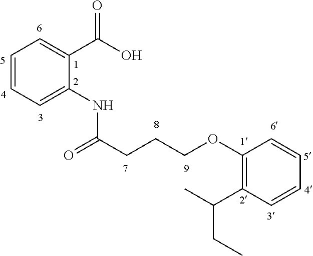 [SMILES: CCC(C)c1ccccc1OCCCC(=O)Nc1ccccc1C(=O)O]

¹³C-NMR: δ [ppm] (101 MHz, DMSO-d₆) 170.7 (CONH), 169.6 (COOH), 155.9 (C-1′), 141.0 (C-2), 134.9 (C-2′), 134.0 (C-4), 131.1 (C-6), 126.6 (C-3′), 126.4 (C-5′), 122.5 (C-5), 120.4 (C-4′), 119.8 (C-3), 116.4 (C-1), 111.6 (C-6′), 66.8 (C-9), 34.2 (C-7), 33.0 (CH), 29.3 (CH₂), 24.6 (C-8), 20.4 (CHCH₃), 12.0 (CH₂CH₃).

IR: \tilde{ν} [cm⁻¹]: 2959, 2925, 2871, 1689, 1606, 1587, 1527, 1492, 1450, 1394, 1379, 1292, 1235, 1163, 1083, 1052, 974, 846, 754, 651, 524.

HRMS (ESI⁺) m/z=calcd for C₂₁H₂₆NO₄: 356.1857 [M+H]⁺, found: 356.1859.

### Example 4

Preparation of

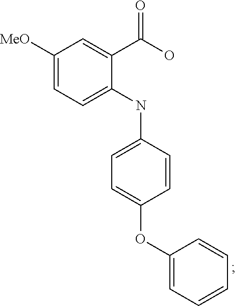

500 mg (2.16 mmol) 2-bromo-5-methoxybenzoic acid and 802 mg (4.33 mmol) 4-phenoxyaniline were dissolved in 5 mL of DMF. 150 mg (1.08 mmol) Potassium carbonate and 14 mg (0.22 mmol) copper powder were added and the reaction was heated to 150° C. for 2 h. The reaction mixture was allowed to reach room temperature and was then added dropwise to 7 mL hydrochloric acid (6 M). The resulting precipitate was collected by filtration with suction and washed with water. The crude product was purified by column chromatography (dichloromethane/methanol). Yield: 144 mg (0.430 mmol, 20%) of a yellow solid.

R_{f} (CH₂Cl₂/CH₃OH 9:1 v/v): 0.53.

MP: 168° C.

¹H-NMR: δ [ppm] (400 MHz, CDCl₃): 8.88 (s, 1H, NH), 7.53 (d, ⁴J_{H,H}=3.0 Hz, 1H, H-6), 7.37-7.31 (m, 2H, H-2′), 7.22-7.17 (m, 2H, H-3″), 7.14 (d, 3J_{H,H}=9.1 Hz, 1H, H-3), 7.12-7.07 (m, 1H, H-4″), 7.06-6.99 (m, 5H, H-4, H-3′, H-2″), 3.81 (s, 3H, OCH₃).

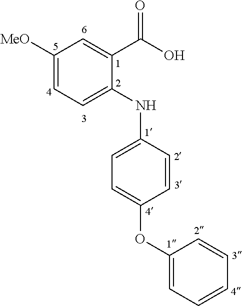 [SMILES: COc1ccc(Nc2ccc(Oc3ccccc3)cc2)c(C(=O)O)c1]

¹³C-NMR: δ [ppm] (101 MHz, CDCl₃): 173.1 (COOH), 157.8 (C-4′), 153.3 (C-1″), 151.1(C-5), 144.1 (C-2), 136.8 (C-1′), 129.9 (C-2′), 124.5 (C-4), 124.4 (C-3″), 123.2 (C-4″), 120.3 (C-2″), 118.5 (C-3′), 116.4 (C-3), 114.2 (C-6), 110.8 (C-1), 56.0 (OCH₃).

IR: \tilde{ν} [cm⁻¹]: 3345, 3041, 2914, 1730, 1644, 1594, 1584, 1516, 1487, 1462, 1441, 1342, 1258, 1217, 1167, 1148, 1070, 1043, 860, 747.

HRMS (ESI⁺) m/z=calcd for C₂₀H₁₈NO₄: 336.1231 [M+H]⁺, found: 336.1232.

### Example 5

Preparation of

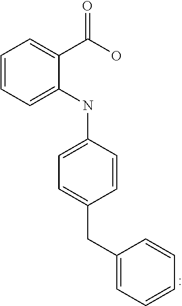 [SMILES: O=C(O)c1ccccc1Nc1ccc(Cc2ccccc2)cc1]

To a solution of 2.02 g (10.0 mmol) 2-bromobenzoic acid in 80 mL methanol were added 2 mL sulphuric acid (conc.) at 0° C. The reaction was heated at reflux for 18 h. After cooling to room temperature the mixture was poured onto ice, neutralized with a saturated aqueous solution of sodium carbonate and extracted with dichloromethane three times. The combined organic layers were dried over sodium sulfate, filtrated and the solvent was evaporated.

Yield: 2.06 g (9.58 mmol, 95%) of a colorless liquid.

200 mg (0.930 mmol) Methyl 2-bromobenzoate and 290 mg (1.58 mmol) 4-benzylaniline were dissolved in 20 mL DME and 128 mg (0.140 mmol) Pd₂(dba)₃, 232 mg (0.372 mmol) rac-BINAP and 334 mg (2.42 mmol) potassium carbonate were added. The reaction was heated at reflux for 16 h. After cooling to room temperature the suspension was diluted with dichloromethane and filtrated. The obtained filtrate was then diluted with water. After separation of the phases the aqueous phase was extracted twice with dichloromethane. The combined organic layers were washed with brine, dried over sodium sulfate, filtrated and the solvent was evaporated. The crude product was purified by column chromatography (petroleum ether 50-70/ethyl acetate).

Yield: 265 mg (0.836 mmol, 90%) of a pale yellow solid.

To a solution of 215 mg (0.677 mmol) methyl 2-(4-benzylphenylamino)benzoate in 13 mL THE 2.5 mL of an aqueous sodium hydroxide solution (1 M) was added. The reaction was heated at reflux for 48 h, and then adjusted to pH 1 with hydrochloric acid (1 M). The reaction was diluted with dichloro-methane and the phases were separated. After extraction of the aqueous phase with dichloromethane twice the combined organic layers were dried over sodium sulfate, filtrated and the solvent was evaporated. The crude product was purified by crystallization from dichloromethane/petroleum ether 50-70.

Yield: 190 mg (0.627 mmol, 93%) of colorless crystals.

R_{f} (CH₂Cl₂/CH₃OH 19:1 v/v): 0.43.

MP: 184° C.

¹H-NMR: δ [ppm] (400 MHz, DMSO-d₆): 13.02 (brs, 1H, COOH), 9.58 (s, 1H, NH), 7.89 (dd, ³J_{H,H}=8.0 Hz, ⁴J_{H,H}=1.6 Hz, 1H, H-6), 7.37-7.13 (m, 11H, H-3, H-4, H-2′, H-3, H-2″, H-3″, H-4″), 6.76-6.71 (m, 1H, H-5), 3.91 (s, 2H, CH₂).

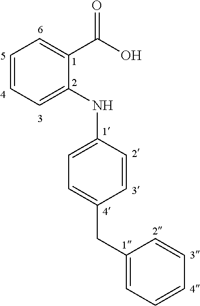 [SMILES: O=C(O)c1ccccc1Nc1ccc(Cc2ccccc2)cc1]

¹³C-NMR: δ [ppm] (101 MHz, DMSO-d₆): 170.0 (COOH), 147.4 (C-2), 141.4 (C-4′), 138.4 (C-1′), 136.3 (C-1″), 134.2 (C-4), 131.8 (C-6), 129.7 (C-3′), 128.7 (C-3″), 128.4 (C-2′), 125.9 (C-4″), 121.9 (C-2″), 117.1 (C-5), 113.5 (C-3), 112.2 (C-1), 40.5 (CH₂).

IR: \tilde{ν} [cm⁻¹]: 3331, 3023, 2838, 1651, 1597, 1573, 1513, 1495, 1421, 1322, 1260, 1161, 1148, 1083, 1042, 888, 751.

HRMS (ESI⁺) m/z=calcd for C₂₀H₁₈NO₂: 304.1332 [M+H]⁺, found: 304.1345.

### Example 6

Preparation of

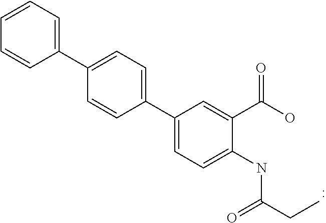 [SMILES: CCC(=O)Nc1ccc(-c2ccc(-c3ccccc3)cc2)cc1C(=O)O]

3.49 g (15.0 mmol) methyl-2-amino-5-bromobenzoate and 10.4 g (75.0 mmol) potassium carbonate were suspended in 300 mL acetone. 3.30 mL (37.5 mmol) propionylchloride were added dropwise at 0° C. and the reaction mixture was stirred at room temperature for 2 hours. After addition of ethyl acetate and a saturated aqueous solution of sodium bicarbonate the phases were separated. The organic phase was washed with demineralised water and brine, dried over sodium sulfate, filtrated and the solvent was evaporated. The product precipitated by addition of petroleum ether 50-70 and was filtrated.

Yield: 3.91 g (13.6 mmol, 91%) of colorless crystals (methyl-5-bromo-2-propionamidobenzoate).

1.2 g (4.0 mmol) methyl-5-bromo-2-propionamidobenzoate, 0.87 g (4.4 mmol) 4-biphenylboronic acid and 0.46 g (0.40 mmol) tetrakis(triphenylphosphine)palladium were dissolved in 2.2 mL ethanol and 10 mL toluene. After addition of 4.0 mL of a 2 M aqueous sodium carbonate solution the reaction mixture was stirred at 100° C. overnight and subsequently diluted with ethyl acetate and a saturated aqueous solution of sodium bicarbonate. The phases were separated, the organic layer was washed with water and brine, dried over sodium sulfate, filtrated and the solvent was evaporated. The crude product was purified by column chromatography (dichloromethane/petroleum ether 50-70).

To a solution of the obtained methyl ester in 8.0 mL THF 3 mL of an aqueous sodium hydroxide solution (1 M) was added. The reaction was stirred at room temperature overnight, and then adjusted to pH 1 with hydrochloric acid (1 M). The reaction was diluted with dichloromethane and the phases were separated. After extraction of the aqueous phase with dichloro-methane twice the combined organic layers were dried over sodium sulfate, filtrated and the solvent was evaporated. The crude product was purified by crystallization from dichloro-methane/petroleum ether 50-70.

Yield over two steps: 1.1 g (3.2 mmol, 80%) of colorless crystals.

R_{f} (CH₂Cl₂/CH₃OH 19:1 v/v): 0.26.

¹H-NMR: δ [ppm] (400 MHz, DMSO-d₆): 13.79 (s, 1H, COCH), 11.15 (s, 1H, CONH), 8.63 (d, ³J_{H,H}=8.8 Hz, 1H, H-5), 8.29 (d, ⁴J_{H,H}=2.4 Hz, 1H, H-2), 7.97 (dd, ³J_{H,H}=8.7 Hz, ⁴J_{H,H}=2.4 Hz, 1H, H-6), 7.77 (s, 4H, H-2′, H-3′), 7.75-7.68 (m, 2H, H-2″), 7.53-7.47 (m, 2H, H-3″), 7.43-7.33 (m, 1H, H-4″), 2.44 (q, ³J_{H,H}=7.5 Hz, 2H, CH₂), 1.15 (t, ³J_{H,H}=7.5 Hz, 3H, CH₃).

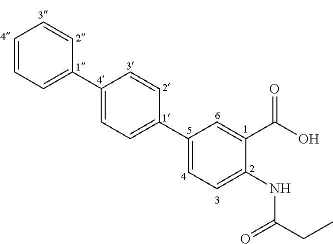 [SMILES: CCC(=O)Nc1ccc(-c2ccc(-c3ccccc3)cc2)cc1C(=O)O]

¹³C-NMR: δ [ppm] (101 MHz, DMSO-d₆): 172.0 (CONH), 169.5 (COOH), 140.3 (C-4), 139.1 (C-1″), 139.1 (C-4′), 137.7 (C-1′), 133.4 (C-1), 132.0 (C-6), 129.0 (C-3″), 128.6 (C-2), 127.6 (C-4″), 127.3 (C-2′/C-3′), 126.8 (C-2′/C-3′), 126.6 (C-2″), 120.5 (C-5), 116.8 (C-3), 30.7 (CH₂), 9.4 (CH₃).

IR: \tilde{ν} [cm⁻¹]: 2876, 2775, 2593, 1707, 1646, 1586, 1483, 1448, 1318, 1212, 834, 798, 764, 730, 695.

HRMS (ESI⁺) m/z=calcd for C₂₂H₂₀NO₃: 346.1438 [M+H]⁺, found: 346.1443.

### Example 7

Preparation of

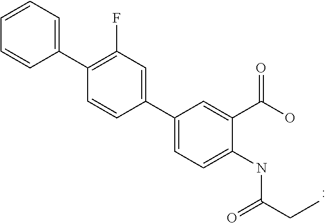 [SMILES: CCC(=O)Nc1ccc(-c2ccc(-c3ccccc3)c(F)c2)cc1C(=O)O]

The compound was synthesized using the procedure following the synthesis of Example 6, with the exception of using 2-fluorobiphenyl-4-boronic acid. 0.30 g (1.0 mmol) methyl-5-bromo-2-propionamidobenzoate, 0.25 g (1.1 mmol) 2-fluorobiphenyl-4-boronic acid and 0.12 g (0.10 mmol) tetrakis(triphenylphosphine)-palladium, 0.55 mL ethanol, 2.2 mL toluene, 1.0 mL of a 2 M aqueous sodium carbonate solution and 2.0 mL of an aqueous sodium hydroxide solution (1 M) were used.

Yield over two steps: 0.13 g (0.35 mmol, 35%) of colorless crystals.

R_{f} (CH₂Cl₂/CH₃OH 19:1 v/v): 0.26.

¹H-NMR: δ [ppm] (600 MHz, DMSO-d₆): 13.82 (s, 1H, COOH), 11.17 (s, 1H, CONH), 8.63 (d, ³J_{H,H}=8.7 Hz, 1H, H-5), 8.29 (d, ⁴J_{H,H}=2.4 Hz, 1H, H-2), 8.00 (dd, ³J_{H,H}=8.7 Hz, ⁴J_{H,H}=2.4 Hz, 1H, H-6), 7.69-7.62 (m, 1H, H-2′), 7.62-7.56 (m, 4H, H-5′, H-6′, H-2″), 7.50 (t, ³J_{H,H}=7.7 Hz, 2H, H-3″), 7.45-7.39 (m, 1H, H-4″), 2.44 (q, ³J_{H,H}=7.5 Hz, 2H, CH₂), 1.14 (t, ³J_{H,H}=7.5 Hz, 3H, CH₃).

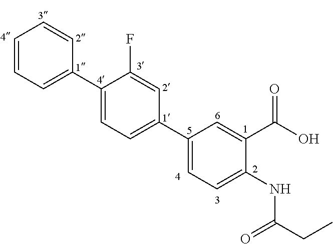 [SMILES: CCC(=O)Nc1ccc(-c2ccc(-c3ccccc3)c(F)c2)cc1C(=O)O]

¹³C-NMR: δ [ppm] (151 MHz, DMSO-d₆): 172.1 (CONH), 169.4 (COOH), 159.5 (d, ¹J_{C,F}=246 Hz, C-3′), 140.7 (C-4), 140.1 (d, ³J_{C,F}=8.3 Hz, C-1′), 134.7 (C-1″) 132.1 (C-1), 131.2 (d, ³J_{C,F}=4.0 Hz, C-5′), 128.8 (C-Aryl), 128.7 (C-Aryl), 128.7 (C-Aryl), 128.6 (C-Aryl), 127.9 (C-4″), 127.0 (d, ²J_{C,F}=13.3 Hz, C-4′), 122.6 (d, ⁴J_{C,F}=4.0 Hz, C-6′), 120.4 (C-5), 116.8 (C-3), 113.7 (d, ²J_{C,F}=23.9 Hz, C-2′), 30.7 (CH₂), 9.3 (CH₃).

¹⁹F-NMR: δ [ppm] (565 MHz, DMSO-d₆): −117.8 (m, Aryl-F).

IR: \tilde{ν} [cm⁻¹]: 3321, 1610, 1558, 1488, 1412, 1386, 1192, 1158, 1135, 906, 823, 788, 764, 694, 671.

HRMS (ESI⁺) m/z=calcd for C₂₂H₁₉FNO₃: 364.1349 [M+H]⁺, found: 346.1351.

### Example 8

Preparation of

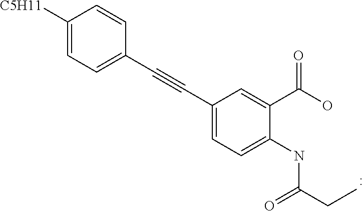

An oven dried microwave vial was charged with 286 mg (1.00 mmol) methyl 5-bromo-2-propionamidobenzoate, 116 mg (0.10 mmol) tetrakis(triphenylphosphine)palladium(0), 38 mg (0.20 mmol) copper(I) iodine and was sealed with a cap. The reaction vessel was evacuated and filled with N₂ followed by 3 mL dry acetonitrile, 1.1 mL of dry triethyl amine and 292 μL (259 mg, 1.50 mmol) 4-ethynyl-pentylbenzene. The reaction was stirred for 1 h at 80° C. (100 Watt), cooled and diluted with dichloro-methane and the solvent was evaporated. The crude product was purified by column chromatography (dichloromethane: 100%). Product containing fraction were pooled and evaporated to dry-ness. Methyl 5-((4-pentylphenyl)ethynyl)-2-propionamidobenzoate was dissolved in 5 mL of THE and treated with 3 mL of an aqueous sodium hydroxide solution (1 M). The reaction was stirred at room temperature for 20 h, and then adjusted to pH 1 with hydrochloric acid (1 M). The reaction was diluted with dichloromethane and the phases were separated. After extraction of the aqueous phase with dichloromethane twice the combined organic layers were dried over sodium sulfate, filtrated and the solvent was evaporated. The crude product was purified by crystallization from dichloromethane/petroleum ether 50-70.

Yield over two steps: 292 mg (0.80 mmol, 80%) of colorless crystals (5-((4-pentylphenyl)ethynyl)-2-propionamidobenzoic acid).

¹H-NMR: δ [ppm] (600 MHz, DMSO-d₆): 13.89 (brs, 1H, COOH), 11.22 (s, 1H, NH), 8.57 (d, 3J_{H,H}=8.7 Hz, 1H, H-3), 8.08 (d, ⁴J_{H,H}=2.1 Hz, 1H, H-6), 7.73 (dd, ³J_{H,H}=8.7 Hz, ⁴J_{H,H}=2.2 Hz, 1H, H-4), 7.49-7.43 (m, 2H, H-2′), 7.27-7.21 (m, 2H, H-3′), 2.59 (t, ³J_{H,H}=8.7 Hz, 2H, H-a), 2.44 (q, ³J_{H,H}=7.5 Hz, 2H, CH₂), 1.57 (p, ³J_{H,H}=7.6 Hz, 2H, H-b), 1.35-1.21 (m, 4H, H-c, H-d), 1.13 (t, ³J_{H,H}=7.5 Hz, 3H, CH₃), 0.86 (t, ³J_{H,H}=7.1 Hz, 3H, H-e).

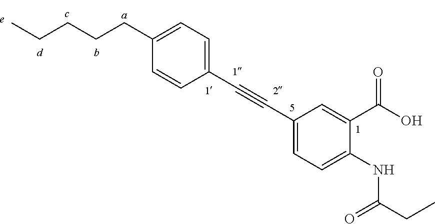 [SMILES: CCCCCc1ccc(C#Cc2ccc(NC(=O)CC)c(C(=O)O)c2)cc1]

¹³C-NMR: δ [ppm] (151 MHz, DMSO-d₆): 172.1 (CONH), 168.8 (COOH), 143.4 (C-4′), 140.8 (C-2), 136.4 (C-4), 133.9 (C-6), 131.3 (C-2′), 128.7 (C-3′), 120.0 (C-3), 119.4 (C-1′), 116.5 (C-1 or C-5), 116.2 (C-1 or C-5), 89.3 (C-1″), 87.7 (C-2″), 34.9 (C-a), 30.8 (C-c), 30.7 (CH₂), 30.3 (C-b), 21.9 (C-d), 13.9 (C-e), 9.23 (CH₃).

HRMS (ESI⁺) m/z=calcd for C₂₃H₂₆NO₃: 364.1907 [M+H]⁺, found: 364.1914.

### Example 9

Preparation of

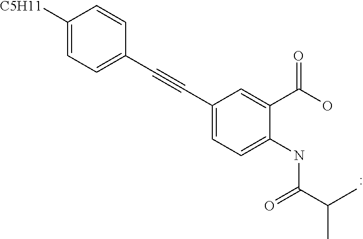

Methyl 5-bromo-2-isobutyramidobenzoate was synthesized using a procedure generally following the synthesis of methyl 5-bromo-2-propionamidobenzoate, with the exception of using 2.60 mL (2.66 g, 25.0 mmol) of isobutyryl chloride instead of propionyl chloride.

Yield: 2.55 g (8.50 mmol, 85%) of colorless crystals (methyl 5-bromo-2-isobutyramidobenzoate).

2-Isobutyramido-5-((4-pentylphenyl)ethynyl)benzoic acid was synthesized using a procedure generally following the synthesis of 5-((4-pentylphenyl)ethynyl)-2-propionamidobenzoic acid, with the exception of using 300 mg (1.00 mmol) of methyl 5-bromo-2-isobutyramidobenzoate instead of methyl 5-bromo-2-propionamidobenzoate.

Yield over two steps: 296 mg (0.78 mmol, 78%) of a colorless amorphous solid (2-isobutyramido-5-((4-pentyl-phenyl) ethynyl)benzoic acid).

¹H-NMR: δ [ppm] (600 MHz, DMSO-d₆): 13.92 (brs, 1H, COOH), 11.30 (s, 1H, NH), 8.58 (d, 3J_{H,H}=8.7 Hz, 1H, H-3), 8.09 (d, ⁴J_{H,H}=2.1 Hz, 1H, H-6), 7.72 (dd, ³J_{H,H}=8.7 Hz, ⁴J_{H,H}=2.1 Hz, 1H, H-4), 7.49-7.42 (m, 2H, H-2′), 7.27-7.19 (m, 2H, H-3′), 2.63-2.54 (m, 3H, H-a, CH(CH₃)₂, 1.56 (p, ³J_{H,H}=7.6 Hz, 1H, H-b), 1.33-1.22 (m (4H, H-c, H-d), 1.18 (d, ³J_{H,H}=6.9 Hz, 6H, CH(CH₃)₂, 0.85 (t, ³J_{H,H}=7.1 Hz, 3H, H-e).

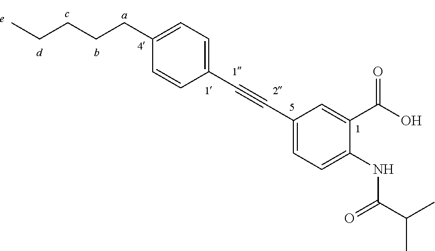 [SMILES: CCCCCc1ccc(C#Cc2ccc(NC(=O)C(C)C)c(C(=O)O)c2)cc1]

¹³C-NMR: δ [ppm] (151 MHz, DMSO-d₆): 175.2 (CONH), 168.9 (COOH), 143.4 (C-4′), 141.0 (C-2), 136.4 (C-4), 133.9 (C-6), 131.3 (C-2′), 128.7 (C-3′), 120.0 (C-3), 119.4 (C-1′), 116.6 (C-1 or C-5), 116.3 (C-5 or C-1) 89.3 (C-1″), 87.7 (C-2″), 36.5 (CH(CH₃)₂), 35.0 (C-a), 30.8 (C-c), 30.3 (C-b), 21.9 (C-d), 19.1 (CH(CH₃)₂), 13.9 (C-e).

HRMS (ESI⁺) m/z=calcd for C₂₄H₂₈NO₃: 378.2064 [M+H]⁺, found: 378.2073.

### Example 10: Assays

Virus Yield Reduction Assay:

The amount of each virus and the duration of the assay have been calibrated by trial so that the replication is still in log phase of growth at the time of readout and the Ct standard deviations of qRT-PCR quantification (quadruplicate) is below 0.5. Approximate multiplicity of infection (MOI) range from 0.001 to 0.1 depending on the strain.

One day prior to infection 5×10⁴ Vero E6 cells were seeded in 100 μl of medium (with 2.5% FCS) in each wells of a 96-well titer plates. The next day, 8 two-fold serial dilutions of the compounds (beginning at 20 μM final concentration, down to 0.16 μM), in triplicates or quadruplicates, were added to the cells (25 μl/well, in 2.5% FCS containing medium). Four Virus Control (VC) wells (per virus) were supplemented with 25 μl medium containing 0.1% DMSO and four cells control wells were supplemented with 50 μl of medium. Fifteen minutes later, 25 μl of a virus mix containing the appropriate amount of viral stock diluted in medium (2.5% FCS) was added to the 96-well plates.

Cells were cultivated for 2 to 4 days after which 100 μl of the supernatant were collected for viral RNA purification. The infected supernatants were transferred to 96 wells S-Bloc from QIAgen preloaded with VXL mix and extract by the Cador Patho-gen 96 QIAcube HT kit run on QIAcube HT automat according to Qiagen protocol. Purified RNAs were eluted in 80 μl of water. Viral RNAs (vRNAs) were then quantified by real time one step RT-PCR to determine viral RNA yield (SuperScript III Platinium one-step RT-PCR from Invitrogen, or GoTaq Probe 1-step RT-PCR system from Promega), using 7.5 μl of RNA and 12.5 μl of RT-PCR mix using standard cycling parameters. The four control wells were replaced by four 2 log dilutions of an appropriate T7-generated RNA standards of known quantities for each viral genome (100 copies to 100 millions copies).

IC50 (Half Maximal Inhibitory Concentration) Determination:

Mean Inhibition of Virus Yield is Equal to:

$$Virus inhibition ( in \% ) = 100 * \frac{\overline{{n}_{vRNA}} ( VC ) - \overline{{n}_{vRNA}} ( drug treated )}{\overline{{n}_{vRNA}} ( VC )}$$

The inhibition values (expressed as percent inhibition, in linear scale) obtained for each drug concentration (expressed in μM, in log scale) are plotted using Kaleidagraph plotting software (Synergy Software) and the best sigmoidal curve, fit-ting the mean values, is determined by a macro in the software: (Inhibition, Y is given by Y=100/1+(m0/m1)^{m2}). This macro allows determining the best curve fit and the m1 and m2 parameters, wherein m1 corresponds to IC50.

Cytotoxicity Assay:

One day prior to the assay 5×10⁴ Vero E6 cells (or 10⁵ HEK 293 cells) were seeded in 100 μl of medium (with 2.5% FCS) in each wells of a 96-well titer plates. The next day, two-fold serial dilutions of the compounds (beginning at 200 μM final concentration, down to 6.2 μM), in triplicates (“drug ex-posed”), were added to the cells (25 μl/well, in 2.5% FCS containing medium). Six cell control (“cell control”) wells were supplemented with 25 μl medium containing two-fold serial dilution of an equivalent amount of DMSO. Eight wells were not seeded by cells and served as background control of fluorescence for the plates (“background control”).

Cells were cultivated for 3 (HEK 293) or 4 (Vero E6) days after which the supernatant was removed and replaced with 70 μl of medium containing CellTiter-Blue reagent (Promega) and further incubated for 90 min at 37° C. Fluorescence (560/590 nm) of the plates indicating reduction of resazurin to resorufin were then read on a TECAN Infinite M 200 Pro reader. The cell viabilities, in percent, were calculated from the fluorescence (F) as:

$$Cell viability ( in \% ) = 100 * \frac{\overline{F} ( drug exposed ) - \overline{F} ( Background control )}{\overline{F} ( cell control ) - \overline{F} ( Background control )}$$

The antiviral activity obtained with the compounds of the examples in simian, mouse, hamster and human cell lines against several RNA virus families is shown the tables below.

| Cell | Virus | | Parameter | Example | | | | |
|---|---|---|---|---|---|---|---|---|
| line | family | Virus | [uM] | 1 | 2 | 3 | 4 | 5 |
| Simian | BUNYA | TOSV | IC₅₀ | 0.09 | 0.55 ± 0.05 | 0.07 ± 0.01 | 0.08 | 0.325 |
| Vero | | | CC₅₀ | 60 ± 15 | 110 ± 10 | 18.5 ± 1.5 | 71 ± 1 | 77.5 |
| E6 | | HAZV | IC₅₀ | 0.05 | 0.7 ± 0.2 | 0.11 | 0.12 | 0.360 |
| | | | CC₅₀ | 60 ± 15 | 110 ± 10 | 18.5 ± 1.5 | 71 ± 1 | 77.5 |
| | | TAHV | IC₅₀ | 0.17 | 0.27 | 0.04 | 0.1 | 0.450 |
| | | | CC₅₀ | 60 ± 15 | 110 ± 10 | 18.5 ± 1.5 | 71 ± 1 | 77.5 |
| | | RVFV | IC₅₀ | 0.09 | ≈2 | 0.16 | 0.02 | n.d.^{a} |
| | | | CC₅₀ | 60 ± 15 | 110 ± 10 | 18.5 ± 1.5 | 71 ± 1 | |
| | FLAVI | YFV | IC₅₀ | 0.2 | 0.7 ± 0.2 | 0.11 | 0.22 | n.d.^{a} |
| | | | CC₅₀ | 60 ± 15 | 110 ± 10 | 18.5 ± 1.5 | 71 ± 1 | 44 |
| | | | | | | | 37.9 | |
| | | DENV | IC₅₀ | 0.19 | n.d.^{a} | 0.11 | 0.18 | n.d.^{a} |
| | | | CC₅₀ | 60 ± 15 | | 18.5 ± 1.5 | 71 ± 1 | |
| | | TBEV | IC₅₀ | 0.22 | n.d.^{a} | 0.08 | 0.12 | n.d.^{a} |
| | | | CC₅₀ | 60 ± 15 | | 18.5 ± 1.5 | 71 ± 1 | |
| | | ZIKV | IC₅₀ | n.d.^{a} | n.d.^{a} | 0.11 | n.d.^{a} | n.d.^{a} |
| | | | CC₅₀ | | | 18.5 ± 1.5 | | |
| | TOGA | VEEV | IC₅₀ | 0.07 | n.d.^{a} | 0.03 | 0.06 | n.d.^{a} |
| | | | CC₅₀ | 60 ± 15 | | 18.5 ± 1.5 | 71 ± 1 | |
| Mouse | BUNYA | RVFV | IC₅₀ | 0.04 | n.d.^{a} | 0.17 | n.d.^{a} | n.d.^{a} |
| L929 | | | CC₅₀ | 60 | | n.d.^{a} | 70 | |
| | FLAVI | TBEV | IC₅₀ | 0.08 | n.d.^{a} | 0.3 | 0.16 | n.d.^{a} |
| | | | CC₅₀ | 60 | | n.d.^{a} | 70 | |
| | TOGA | VEEV | IC₅₀ | n.d.^{a} | n.d.^{a} | 0.6 | 0.22 | >1 |
| | | | CC₅₀ | | | n.d.^{a} | 70 | 60 |
| Hamster | BUNYA | RVFV | IC₅₀ | 0.7 ± 0.03 | n.d.^{a} | n.d.^{a} | 0.85 | n.d.^{a} |
| BHK-21 | | | CC₅₀ | 140 | | | 125 | |
| | FLAVI | YFV | IC₅₀ | n.d.^{a} | n.d.^{a} | n.d.^{a} | n.d.^{a} | n.d.^{a} |
| | | | CC₅₀ | | | | 125 | |
| | | TBEV | IC₅₀ | n.d.^{a} | n.d.^{a} | n.d.^{a} | n.d.^{a} | n.d.^{a} |
| | | | CC₅₀ | | | | 125 | |
| | TOGA | VEEV | IC₅₀ | 0.36 | n.d.^{a} | n.d.^{a} | 1.3 | n.d.^{a} |
| | | | CC₅₀ | 140 | | | 125 | |
| Human | BUNYA | RVFV | IC₅₀ | 0.28 | 0.5 | 0.05 | 0.13 | n.d.^{a} |
| SW13 | | | CC₅₀ | 60 | n.d.^{a} | n.d.^{a} | 75 | |
| | FLAVI | TBEV | IC₅₀ | 0.67 | 0.93 | 0.1 | 0.38 | n.d.^{a} |
| | | | CC₅₀ | 60 | n.d.^{a} | n.d.^{a} | 75 | |
| | TOGA | VEEV | IC₅₀ | 0.1 | 1.5 | 0.13 | 0.22 | n.d.^{a} |
| | | | CC₅₀ | 60 | n.d.^{a} | n.d.^{a} | 75 | |
| Human | BUNYA | RVFV | IC₅₀ | 0.04 ± 0.02 | 0.4 | 0.015 | n.d.^{a} | n.d.^{a} |
| HEK-293 | | | CC₅₀ | 80 | n.d.^{a} | n.d.^{a} | 125 | |
| | TOGA | VEEV | IC₅₀ | n.d.^{a} | 0.45 | 0.05 | n.d.^{a} | n.d.^{a} |
| | | | CC₅₀ | | n.d.^{a} | n.d.^{a} | | |

^{a}not determined,

| Cell | Virus | | Parameter | Example | | | |
|---|---|---|---|---|---|---|---|
| line | family | Virus | [uM] | 6 | 7 | 8 | 9 |
| Simian | BUNYA | TOSV | IC₅₀ | 0.12 | 0.11 | 0.01 | 0.01 |
| Vero | | | CC₅₀ | 10 | 15 ± 0 | 8 | 4 |
| E6 | | HAZV | IC₅₀ | 0.12 | 0.07 | 0.008 | 0.012 |
| | | | CC₅₀ | 10 | 15 ± 0 | 8 | 4 |
| | | TAHV | IC₅₀ | n.d.^{a} | 0.12 | 0.01 | n.d.^{a} |
| | | | CC₅₀ | | 15 ± 0 | 8 | |
| | | RVFV | IC₅₀ | 0.14 | 0.12 | 0.006 | 0.01 |
| | | | CC₅₀ | 10 | 15 ± 0 | 8 | 4 |
| | FLAVI | YFV | IC₅₀ | n.d.^{a} | n.d.^{a} | 0.006 | 0.016 |
| | | | CC₅₀ | | | 8 | 4 |
| | | DENV | IC₅₀ | n.d.^{a} | n.d.^{a} | 0.005 | 0.023 |
| | | | CC₅₀ | | | 8 | 4 |
| | | TBEV | IC₅₀ | 0.12 ± 0.01 | 0.013 | 0.07 | 0.0117 |
| | | | CC₅₀ | 10 | 15 ± 0 | 8 | 4 |
| | | ZIKV | IC₅₀ | n.d.^{a} | n.d.^{a} | 0.009 | 0.023 |
| | | | CC₅₀ | | | 8 | 4 |
| | TOGA | VEEV | IC₅₀ | 0.12 | 0.12 | 0.007 | 0.0107 |
| | | | CC₅₀ | 10 | 15 | 8 | 4 |
| Mouse | BUNYA | RVFV | IC₅₀ | 0.003 | 0.0055 | 0.002 | n.d.^{a} |
| L929 | | | CC₅₀ | 15 | 9 | 5 | |
| | FLAVI | TBEV | IC₅₀ | 0.009 | 0.004 | 0.0015 | n.d.^{a} |
| | | | CC₅₀ | 15 | 9 | 5 | |
| | TOGA | VEEV | IC₅₀ | 0.009 | 0.007 | 0.003 | n.d.^{a} |
| | | | CC₅₀ | 15 | 9 | 5 | |
| Hamster | BUNYA | RVFV | IC₅₀ | >0.2 | n.d.^{a} | 0.016 | 0.043 |
| BHK-21 | | | CC₅₀ | n.d.^{a} | | 7.5 | 50 |
| | FLAVI | YFV | IC₅₀ | >0.2 | n.d.^{a} | 0.022 | 0.055 |
| | | | CC₅₀ | n.d.^{a} | | 7.5 | 55 |
| | | TBEV | IC₅₀ | n.d.^{a} | n.d.^{a} | n.d.^{a} | n.d.^{a} |
| | | | CC₅₀ | | | 7.5 | 55 |
| | TOGA | VEEV | IC₅₀ | n.d.^{a} | n.d.^{a} | n.d.^{a} | n.d.^{a} |
| | | | CC₅₀ | | | 7.5 | 55 |
| Human | BUNYA | RVFV | IC₅₀ | n.d.^{a} | n.d.^{a} | 0.008 | 0.011 |
| SW13 | | | CC₅₀ | | | 20 | 17 |
| | FLAVI | TBEV | IC₅₀ | n.d.^{a} | n.d.^{a} | 0.013 | 0.014 |
| | | | CC₅₀ | | | 20 | 17 |
| | TOGA | VEEV | IC₅₀ | n.d.^{a} | n.d.^{a} | 0.013 | 0.014 |
| | | | CC₅₀ | | | 20 | 17 |
| Human | BUNYA | RVFV | IC₅₀ | n.d.^{a} | n.d.^{a} | 0.0015 | <<0.0016 |
| HEK-293 | | | CC₅₀ | | | n.d.^{a} | n.d.^{a} |
| | FLAVI | TBEV | IC₅₀ | n.d.^{a} | n.d.^{a} | 0.0031 | 0.0044 |
| | | | CC₅₀ | | | n.d.^{a} | n.d.^{a} |
| | TOGA | VEEV | IC₅₀ | 0.019 | 0.014 | 0.0015 | 0.0015 |
| | | | CC₅₀ | n.d.^{a} | n.d.^{a} | n.d.^{a} | n.d.^{a} |

^{a}not determined

### Example 11: ADME Properties

Absorption, distribution, metabolism and excretion properties of selected inhibitors were determined and shown in the following table.

| | Ex. 1 | Ex. 3 | Ex. 6 | Ex. 8 | Ex. 9 |
|---|---|---|---|---|---|
| Solubility (PBS | 167 ± 7 | 1540 ± 50 | 61 ± 17 | 18 ± 6 | 13 ± 6 |
| pH 7.4) [μM]^{a} | | | | | |
| Plasma protein | 99 ± 0 | 99 ± 0 | 100 ± 0 | n.d. | n.d. |
| binding [%]^{b} | | | | | |
| Log D (PBS pH | 2.62 ± 0.03 | 2.59 ± 0.03 | 3.07 ± 0.05 | 4.6 ± 0.3 | n.d. |
| 7.4/octanol) | | | | | |
| Hepatic | n.d. | n.d. | 49 ± 0 | 70 ± 3 | 45 ± 8 |
| microsome | | | | | |
| stability [% | | | | | |
| remaining at 1 | | | | | |
| h]^{d} | | | | | |
| Plasma stability | 75 ± 0 | 85 ± 4 | 94 ± 6 | 114 ± 5 | 98 ± 3 |
| [% remaining at | | | | | |
| 2 h]^{b} | | | | | |

^{a}at ambient temperature (20° C.)
^{b}1 μm, 37° C., mouse plasma diluted with 1 volume of PBS pH 7.4
^{c}PBS pH 7.4, 5% DMSO; ambient temperature; membrane: dodecane 2% phosphatidylcholine
^{d}1 μm, 37° C., 0.5 mg/mL microsomes of mouse liver; cofactor: NADPH

The above data show that the tested compounds have acceptable ADME properties. The 5-ethynyl derivative of Example 8 is more stable than the compound of Example 6, but has lower solubility.

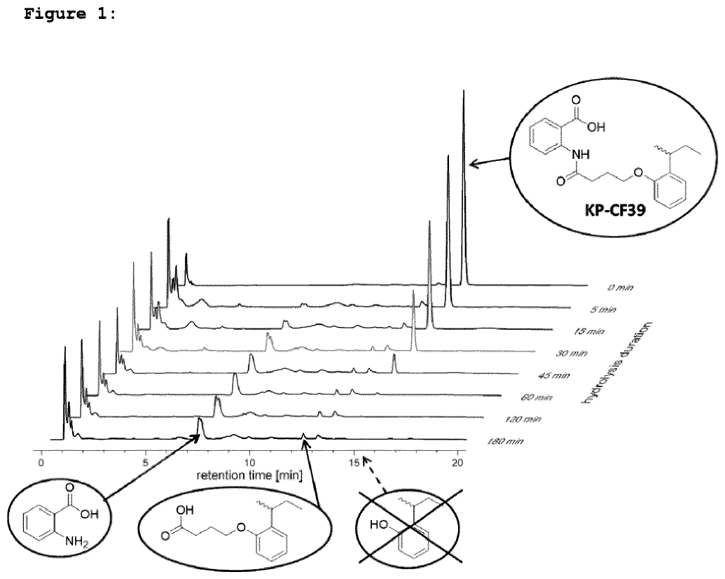

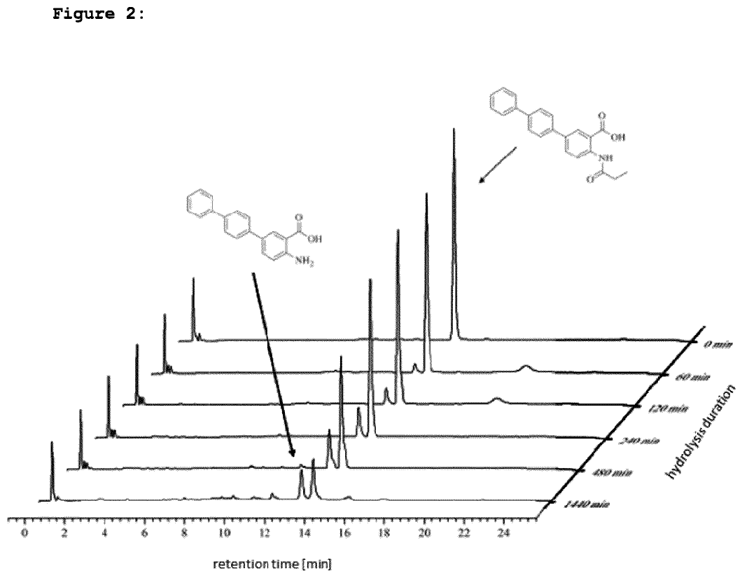

A hydrolysis study of the compound of Example 3 in mouse liver extract is shown in FIG. 1. The results of this test show a cleavage of the amide bond, while the ether bond remained in-tact. Apparently, the amide bond in Example 6 is more stable (see FIG. 2).

### Example 12

Preparation of

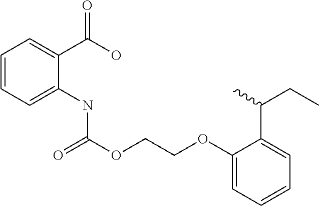 [SMILES: CCC(C)c1ccccc1OCCOC(=O)Nc1ccccc1C(=O)O]

The compound of Example 12 was synthesized in accordance with Scheme 15.

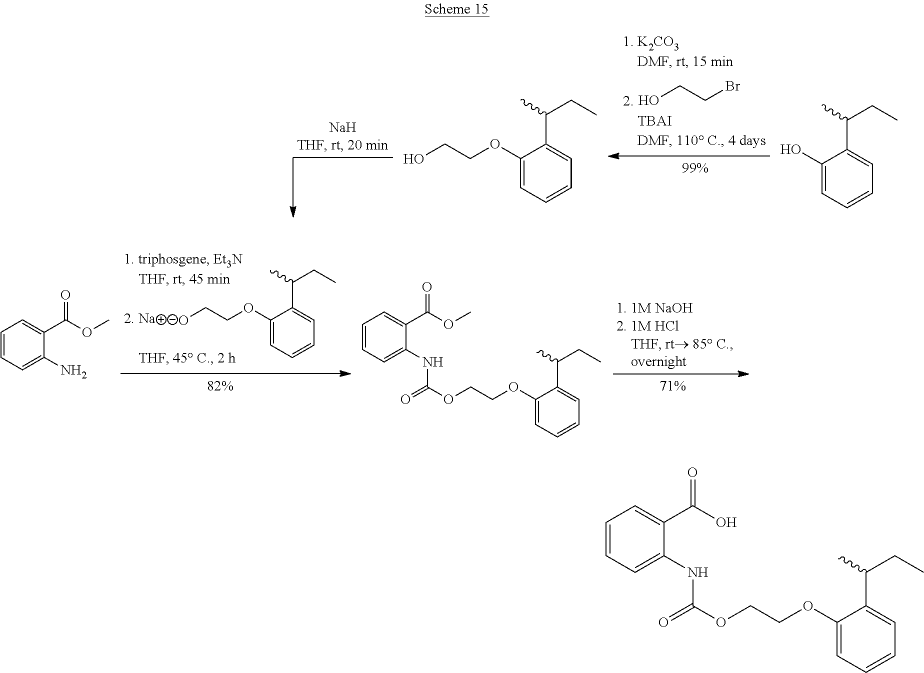

The carbamate compound of Example 12 has been found to have an IC₅₀ of 190 nM (TOSV) and a CC₅₀ of 51 μM. It is characterized by a high metabolic stability in a S9 fraction (rat).

### Example 13

Preparation of

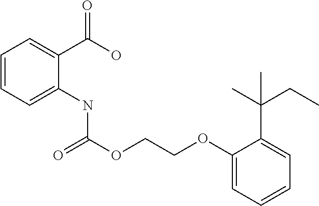 [SMILES: CCC(C)(C)c1ccccc1OCCOC(=O)Nc1ccccc1C(=O)O]

The compound of Example 13 was synthesized in accordance with Scheme 16.

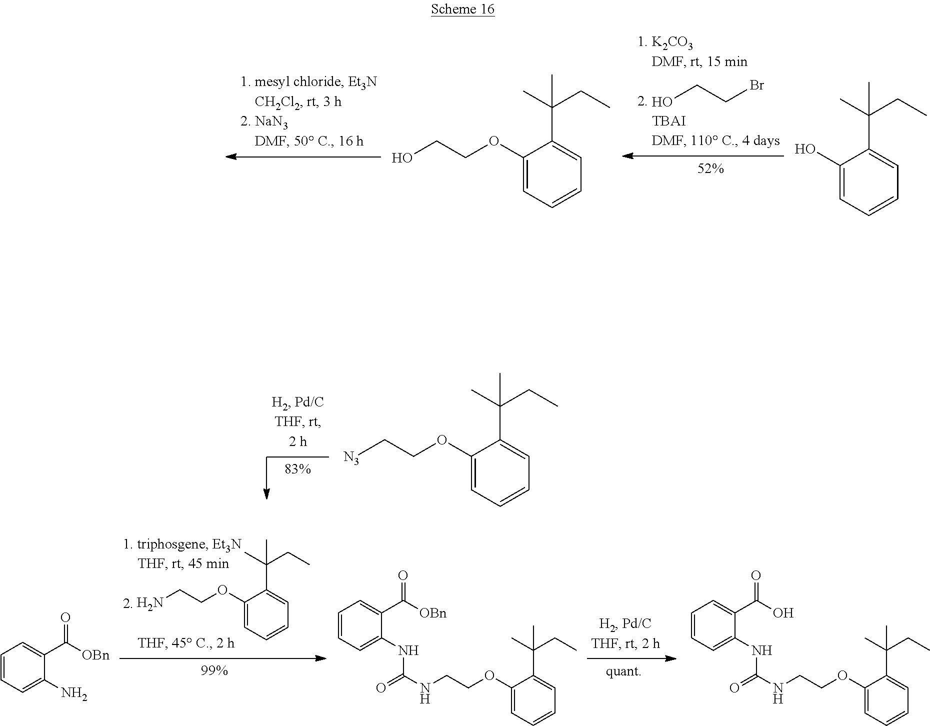

The urea compound of Example 13 has been found to have an IC₅₀ of 128 nM (TOSV) and a CC₅₀ of more than 25 μM. It is characterized by a high metabolic stability in a S9 fraction (rat).

### Example 14

Preparation of

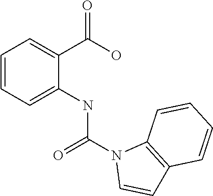 [SMILES: O=C(O)c1ccccc1NC(=O)n1ccc2ccccc21]

The compound of Example 14 was synthesized in accordance with Scheme 17.

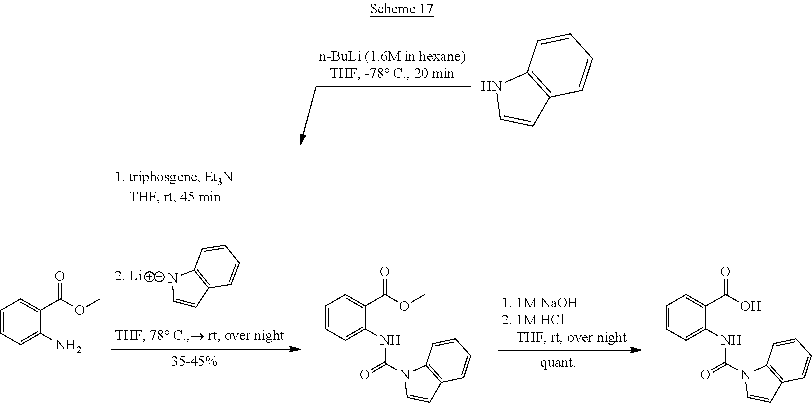 [SMILES: c1ccc2[nH]ccc2c1 ; COC(=O)c1ccccc1N ; c1ccc2[n-]ccc2c1 ; COC(=O)c1ccccc1NC(=O)n1ccc2ccccc21 ; O=C(O)c1ccccc1NC(=O)n1ccc2ccccc21]

The compound of Example 14 has been found to have an IC₅₀ of 9 μM (TOSV) and a CC₅₀ of 40 μM.

### Example 15

Preparation of

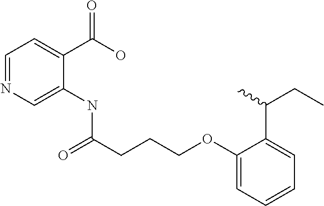 [SMILES: CCC(C)c1ccccc1OCCCC(=O)Nc1cnccc1C(=O)O]

The compound of Example 15 was synthesized in accordance with Scheme 1. It has been found to have an IC₅₀ of about 0.5 μM (TOSV) and a CC₅₀ of more than 25 μM.

### Example 16

Preparation of

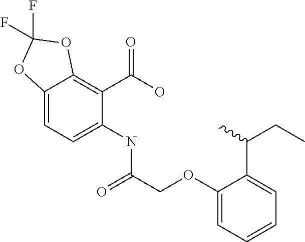 [SMILES: CCC(C)c1ccccc1OCC(=O)Nc1ccc2c(c1C(=O)O)OC(F)(F)O2]

The compound of Example 16 was synthesized in accordance with Scheme 1. It has been found to have an IC₅₀ of 4 μM (TOSV) and a CC₅₀ of 37 μM.

### Example 17

Preparation of

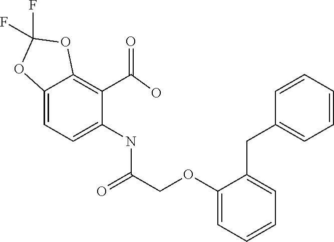 [SMILES: O=C(COc1ccccc1Cc1ccccc1)Nc1ccc2c(c1C(=O)O)OC(F)(F)O2]

The compound of Example 17 was synthesized in accordance with Scheme 1. It has been found to have an IC₅₀ of 6 μM (TOSV) and a CC₅₀ of 32 μM.

### Example 18

Preparation of

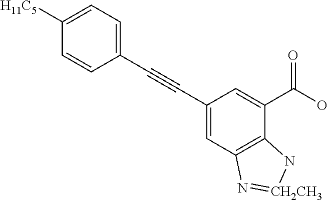

The compound of Example 18 was synthesized in accordance with Scheme 14. It has been found to have an IC₅₀ of 123 nM (TOSV) and a CC₅₀ of more than 25 μM.

### Example 19

Preparation of

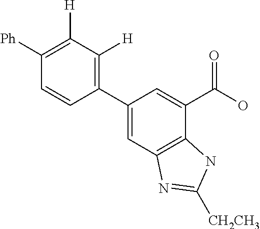 [SMILES: CCc1nc2cc(-c3ccc(-c4ccccc4)cc3)cc(C(=O)O)c2[nH]1]

The compound of Example 19 was synthesized in accordance with Scheme 14. It has been found to have an IC₅₀ of 118 nM (TOSV) and a CC₅₀ of 8 μM.

### Example 20

Preparation of

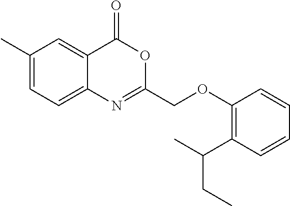 [SMILES: CCC(C)c1ccccc1OCc1nc2ccc(C)cc2c(=O)o1]

The compound of Example 20 was synthesized in accordance with Scheme 11. It has been found to have an IC₅₀ of 0.6 μM (TOSV) and a CC₅₀ of 75 μM.

### Example 21

Preparation of

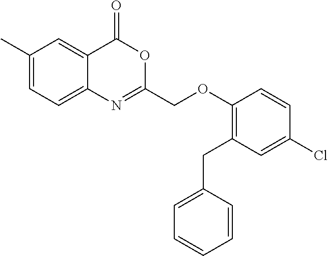 [SMILES: Cc1ccc2nc(COc3ccc(Cl)cc3Cc3ccccc3)oc(=O)c2c1]

The compound of Example 21 was synthesized in accordance with Scheme 11. It has been found to have an IC₅₀ of 0.8 μM (TOSV) and a CC₅₀ of more than 100 μM.

### Example 22

Preparation of

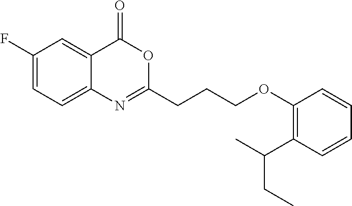 [SMILES: CCC(C)c1ccccc1OCCCc1nc2ccc(F)cc2c(=O)o1]

The compound of Example 22 was synthesized in accordance with Scheme 11. It has been found to have an IC₅₀ of 0.3 μM (TOSV) and a CC₅₀ of 17 μM.

### Example 23

Preparation of

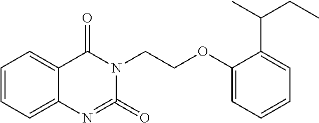

The compound of Example 23 was synthesized in accordance with Scheme 12. It has been found to have an IC₅₀ of 6 μM (TOSV) and a CC₅₀ of more than 200 μM.

### Example 24

Preparation of

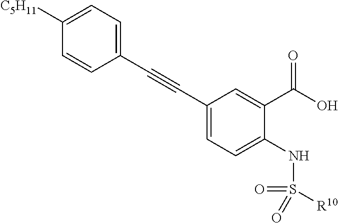

The compounds of Example 24 were synthesized in accordance with Scheme 18 with R¹⁰ being methyl, ethyl, n-propyl, n-butyl, iso-propyl and iso-butyl.

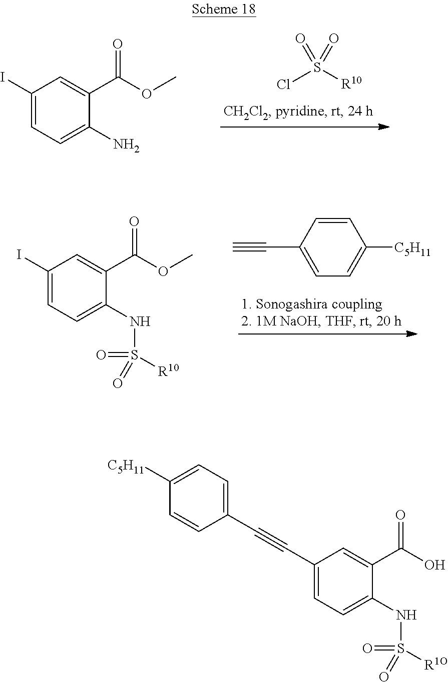

The obtained yield was 23 to 51% (first step of Scheme 18) and 65 to 79% (second step of Scheme 18), respectively. The methyl sulfonamide (R¹⁰ being methyl) has been found to have an IC₅₀ of 1.5 μm (TOSV).

### Example 25

Preparation of

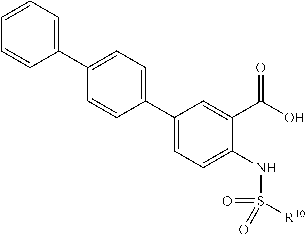

The compounds of Example 25 can be synthesized in accordance with Scheme 19 with R¹⁰ being as defined in Example 24.

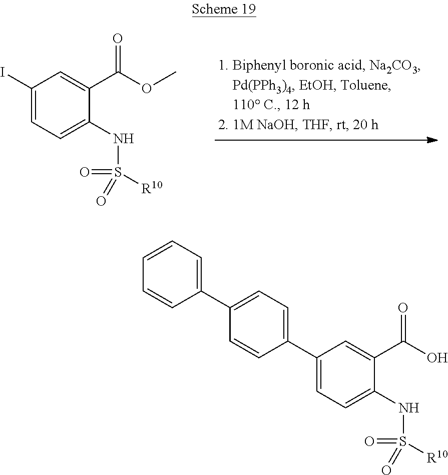

1. Compound, or a dimer or a pharmaceutically acceptable salt or solvate of said compound or dimer, for use in a method for the treatment of a disease, disorder or condition caused by an RNA virus, said compound having the general structure shown in Formula I: 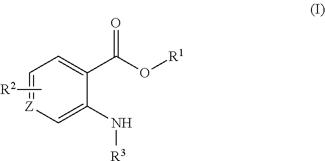
wherein:
Z is C or N, and preferably is C;
R¹ is H, alkyl, cycloalkyl, heterocyclyl, —C(O)-alkyl or a pharmaceutically acceptable cation, wherein the alkyl or
—C(O)-alkyl can be unsubstituted or optionally substituted with one or more moieties which can be the same or different, each moiety being independently selected from the group consisting of —OC(O)-alkyl, —OC(O)O-alkyl, heterocyclyl, aryl and heteroaryl,
or optionally the —C(O)—O—R¹-group is joined to the —NH—R³-group to form together with the aromatic ring shown in Formula (I) a hetero ring system;
R² is one or more substituents independently selected from the group consisting of H, alkyl, alkenyl, alkynyl, aryl, alkoxy, aryloxy, halogen, haloalkyl, hydroxyalkyl and —NO₂, wherein each of said alkyl, alkenyl, alkynyl, aryl, alkoxy and aryloxy can be unsubstituted or optionally substituted with one or more moieties which can be the same or different, each moiety being independently selected from the group consisting of hydroxyl, halogen, alkyl, haloalkyl, aryl, haloaryl, alkylaryl, arylalkyl, cycloalkyl, aryloxy, alkoxy substituted with aryl, alkyl substituted with heterocyclyl, aryl substituted with haloaklkyl, aryl substituted with cycloalkyl, aryl substituted with arylalkyl, aryl substituted with alkoxy, aryl substituted with aryloxy, aryl substituted with —O-arylalkyl, aryl substituted with aryl, aryloxy, aryloxy substituted with alkyl, aryloxy substituted with cycloalkyl and aryloxy substituted with arylalkyl,
or optionally R² represents two substituents which are joined to form together with the aromatic ring shown in Formula I a substituted or unsubstituted ring or hetero ring system, or optionally at least one of R² is joined to the —NH—R³-group to form together with the aromatic ring shown in Formula (I) a hetero ring system;
R³ is —C(O)-alkyl, —C(O)O-alkyl, —C(O)NH-alkyl, —C(O)-cycloalkyl, —C(O)O-cycloalkyl, —C(O)NH-cycloalkyl, —C(O)-aryl, —C(O)O-aryl, —C(O)NH-aryl, —C(O)-heteroaryl, —C(O)O— heteroaryl, —C(O)NH-heteroaryl, aryl substituted with R⁵, heteroaryl substituted with R⁵, —S(O₂)—R⁹ or 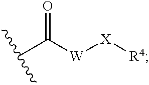
wherein
W is —(CH₂)ₙ—, —O—(CH₂)ₙ—, —NH—(CH₂)ₙ—, —(CH₂)ₚ-L-(CH₂)_{q}—,
—O—(CH₂)ₚ-L-(CH₂)_{q}— or —NH—(CH₂)ₚ-L-(CH₂)_{q}—,
n is an integer from 1 to 6;
p and q are integers independently selected from 0 to 6;
L is a linking group selected from the group consisting of heteroaryl, aryl, heterocyclyl and cycloalkyl;
X is O, S, NH, CH₂, S(O) or C(O), wherein X preferably is O or S or NH or CH₂;
R⁴ is aryl or heteroaryl, wherein said aryl or heteroaryl can be unsubstituted or optionally substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl;
R⁵ is aryloxy or arylalkyl or optionally R⁵ represents two substituents linked to each other to form together with the aryl or heteroaryl a polycyclic ring system; and
R⁹ is alkyl, cycloalkyl or —W—X—R⁴.

2. Compound according to claim 1, or a dimer or a pharmaceutically acceptable salt or solvate of said compound or dimer, wherein
R¹ is selected from the group consisting of H, 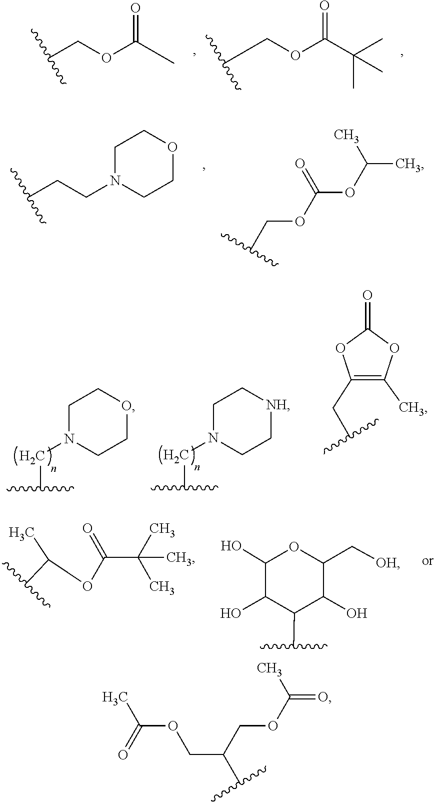
wherein R¹ is preferably selected from the group consisting of H, 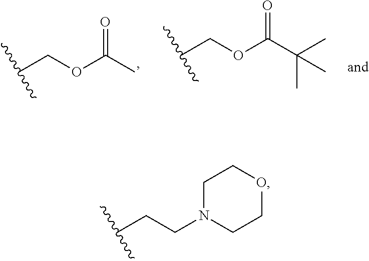
and more preferably is H.

3. Compound according to claim 1, or a dimer or a pharmaceutically acceptable salt or solvate of said compound or dimer, wherein
R¹ is joined to the —NH—R³-group to form together with the aromatic ring shown in Formula (I) a hetero ring system, wherein said hetero ring system preferably comprises an oxazine or quinazolinone moiety so that the compound is represented by 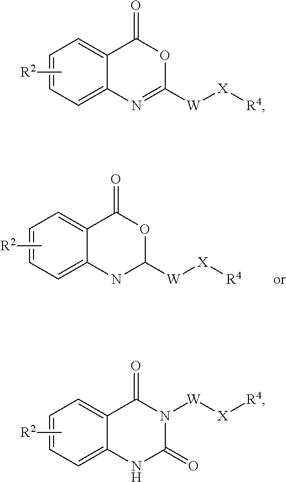
with W preferably being —(CH₂)ₙ—.

4. Compound according to claim 1, or a dimer or a pharmaceutically acceptable salt or solvate of said compound or dimer, wherein
R² is aryl, preferably phenyl, which can be unsubstituted or optionally substituted with one or more moieties which can be the same or different, each moiety being independently selected from the group consisting of alkyl, aryl, halogen, haloaryl, alkylaryl, arylalkyl, cycloalkyl, aryloxy, alkoxy substituted with aryl, alkyl substituted with heterocyclyl.

5. Compound according to claim 4, or a dimer or a pharmaceutically acceptable salt or solvate of said compound or dimer, wherein
R² is 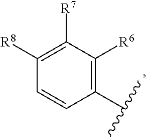
wherein
R⁶ is H; halogen, preferably F; or aryl, preferably phenyl;
R⁷ is H; halogen, preferably F; alkyl, preferably methyl; or aryl, preferably phenyl; and
R⁸ is H; cycloalkyl, preferably cyclohexyl; aryl, preferably phenyl; haloaryl, preferably 4-F-phenyl; arylalkyl, preferably 4-ethyl-phenyl, 4-pentyl-phenyl; alkylaryl, preferably benzyl; aryloxy, preferably phenyloxy; arylalkoxy, preferably benzyloxy; or heterocyclylalkyl, preferably morpholinomethyl.

6. Compound according to claim 1, or a dimer or a pharmaceutically acceptable salt or solvate of said compound or dimer, wherein
R² is alkynyl, preferably ethynyl, which can be unsubstituted or optionally substituted with a moiety selected from the group consisting of aryl, aryl substituted with alkyl, aryl substituted with haloaklkyl, aryl substituted with cycloalkyl, aryl substituted with arylalkyl, aryl substituted with alkoxy, aryl substituted with aryloxy, aryl substituted with —O— arylalkyl, aryl substituted with aryl, aryloxy, aryloxy substituted with alkyl, aryloxy substituted with cycloalkyl and aryloxy substituted with arylalkyl.

7. Compound according to claim 6, or a dimer or a pharmaceutically acceptable salt or solvate of said compound or dimer, wherein
R² is alkynyl, preferably ethynyl, which is substituted with a moiety selected from the group consisting of phenyl; phenyl substituted with 4-haloalkyl like 4-CF₃; phenyl substituted with 4-alkyl like 4-C₄H₉ or 4-C₆H₁₃; phenyl substituted with 4-alkoxy like 4-ethoxy or 4-pentoxy; phenyl substituted with 4-aryloxy like 4-phenyloxy; phenyl substituted with arylalkoxy like 4-benzyloxy; phenyl substituted with 4-aryl like 4-phenyl; phenyl substituted with 4-cycloalkyl like 4-cyclohexyl; phenyl substituted with 4-arylalkyl like 4-benzyl; and alkyl, preferably methyl or butyl, which is substituted with aryloxy, preferably phenyloxy, which in turn is substituted with 2-alkyl, like 2-sec-butyl, 2-cycloalkyl, like 2-cyclohexyl, or 2-arylalkyl, like 2-benzyl.

8. Compound according to claim 1, or a dimer or a pharmaceutically acceptable salt or solvate of said compound or dimer, wherein
R² is H; alkyl, preferably 5-alkyl like 5-methyl or 5-tBu; halogen, preferably 5-halogen like 5-Br or 5-F or 4-halogen like 4-F; alkoxy, preferably 5-alkoxy like 5-methoxy or 4-alkoxy like 4-methoxy; haloalkyl, preferably 5-CF₃; NO₂, preferably 5-NO₂; or aryl, preferably phenyl or biphenyl like 5-phenyl or 5-biphenyl.

9. Compound according to claim 1, or a dimer or a pharmaceutically acceptable salt or solvate of said compound or dimer, wherein
R² represents two substituents which are joined to form together with the aromatic ring shown in Formula (I) a substituted or unsubstituted ring or hetero ring system, wherein said hetero ring system is 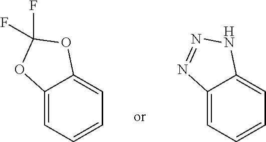 [SMILES: FC1(F)Oc2ccccc2O1 ; c1ccc2[nH]nnc2c1]
so that the compound is preferably represented by

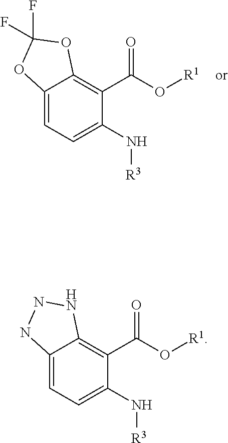

10. Compound according to claim 1, or a dimer or a pharmaceutically acceptable salt or solvate of said compound or dimer, wherein
at least one of R² is joined to the
—NH—R³-group to form together with the aromatic ring shown in Formula (I) a hetero ring system, wherein said hetero ring system preferably comprises a benzimidazole moiety, so that the compound is represented by 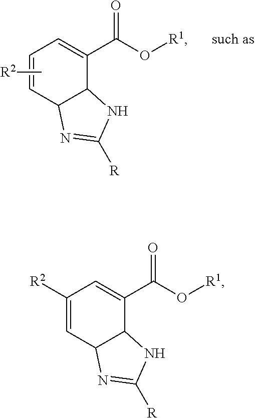
with R being e.g. alkyl.

11. Compound according to claim 1, or a dimer or a pharmaceutically acceptable salt or solvate of said compound or dimer, wherein R³ is
(i) —C(O)-alkyl, —C(O)—O-alkyl or —C(O)—NH-alkyl, and preferably —C(O)-alkyl, with alkyl being methyl, ethyl or isopropyl, preferably ethyl;
(ii) aryl, preferably phenyl, substituted with R⁵, with R⁵ being benzyl or phenoxy; 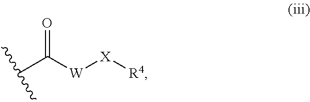
with W preferably being —(CH₂)ₙ—, —O—(CH₂)ₙ— or —NH—(CH₂)ₙ—, and more preferably being —(CH₂)ₙ—, and n being 1 to 6, preferably 1 to 3, more preferably 1;
X being O or S or CH₂ or SO or CO, and preferably O or S or CH₂, and more preferably O; and
R⁴ preferably being phenyl substituted with 2-sec-butyl, 4-sec-butyl, 2-tert-amyl, 2-tert-butyl, 4-tert-butyl, 2-tert-butyl-4-methyl, 2,6-di-tert-butyl-4-methyl, 2-methyl, 2,6-dimethyl, 3,5-dimethyl, 2,4-dimethyl, 2,3,5-trimethyl, 2,4,6-trimethyl, 2-isopropyl, 2-methyl-5-isopropyl, 5-methyl-2-isopropyl, 2,6-di-isopropyl, 2-ethyl, 2-propyl, 2-ethoxy, 2-cyclohexyl, 2-cyclopentyl, 2-adamantanyl-4-methyl, 2-benzyl, 2-benzyl-4-chloro, 2-phenyl, 3-phenyl, 4-phenyl, 1-naphthyl, 2-naphthyl, 4-phenoxy, 2,6-dichloro, 2-iodo or 2-bromo-4-methyl; 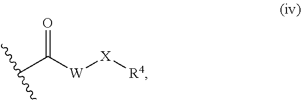
with W preferably being —(CH₂)ₚ-L-(CH₂)_{q}—, —O—(CH₂)ₚ-L-(CH₂)_{q}— or —NH—(CH₂)ₚ-L-(CH₂)_{q}—, with the linking group L preferably being heteroaryl and more preferably 1,4-triazole or 1,5-triazole, and p and q being 0 to 6, preferably 0 to 4, and more preferably 1 to 4, such as 1 to 2;
X being O or S or CH₂, and preferably O; and
R⁴ preferably being phenyl substituted with 2-sec-butyl, 4-sec-butyl, 2-tert-amyl, 2-tert-butyl, 4-tert-butyl, 2-tert-butyl-4-methyl, 2,6-di-tert-butyl-4-methyl, 2-methyl, 2,6-dimethyl, 3,5-dimethyl, 2,4-dimethyl, 2,3,5-trimethyl, 2,4,6-trimethyl, 2-isopropyl, 2-methyl-5-isopropyl, 5-methyl-2-isopropyl, 2,6-di-isopropyl, 2-ethyl, 2-propyl, 2-ethoxy, 2-cyclohexyl, 2-cyclopentyl, 2-adamantanyl-4-methyl, 2-benzyl, 2-benzyl-4-chloro, 2-phenyl, 3-phenyl, 4-phenyl, 1-naphthyl, 2-naphthyl, 4-phenoxy, 2,6-dichloro, 2-iodo or 2-bromo-4-methyl;
(v) —C(O)-heteroaryl, wherein the heteroaryl can be unsubstituted or substituted and preferably is N-pyrrole, N-indole or N-carbazole;
(vi) —C(O)—NH-aryl, wherein the aryl can be unsubstituted or substituted, wherein the aryl preferably is unsubstituted and/or wherein the aryl preferably is phenyl; or
(vii) —S(O₂)—R⁹ with R⁹ being alkyl.

12. Compound according to claim 1, or a dimer or a pharmaceutically acceptable salt or solvate of said compound or dimer, wherein the RNA virus is selected form the group consisting of bunya viruses including Toscana virus (TOSV), hazara virus (HAZV), tahyna virus (TAHV), rift valley fever virus (RVFV), Lassa virus (LSAV), Punta Toro phlebovirus (PTV) and Crimean-Congo hemorrhagic fever orthonairovirus (CCHFV); flavi viruses including yellow fever virus (YFV), dengue virus (DENV), tick-borne encephalitis virus (TBEV), zika virus (ZIKV) and Hepatitis C virus (HCV); toga viruses including venezuelan equine encephalitis virus (VEEV), Sindbis virus (SINV) and Chikungunya virus (CHIKV); mononegaviruses including Ebola virus (EBOV), Marburg virus (MARV), Human parainfluenza virus 3 (HPIV-3), Nipah virus (NiV) and Vesicular stomatitis virus (VSV); picorna viruses including coxsackievirus (CV); nidoviruses including Severe acute respiratory syndrome-related coronavirus (SARS-CoV), Severe acute respiratory syndrome-related coronavirus 2 (SARS-CoV-2) and Middle-East respiratory syndrome-related coronavirus (MERS-CoV); and reoviruses including reovirus type 1 (Reo-1).

13. Compound, or a dimer or a pharmaceutically acceptable salt or solvate of said compound or dimer, having the general structure shown in Formula I: 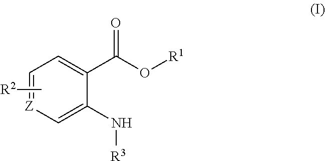
wherein:
Z is C or N, and preferably is C;
R¹ is H, alkyl, cycloalkyl, heterocyclyl, —C(O)-alkyl or a pharmaceutically acceptable cation, wherein the alkyl or
—C(O)-alkyl can be unsubstituted or optionally substituted with one or more moieties which can be the same or different, each moiety being independently selected from the group consisting of —OC(O)-alkyl, —OC(O)O-alkyl, heterocyclyl, aryl and heteroaryl,
or optionally the —C(O)—O—R¹-group is joined to the —NH—R³-group to form together with the aromatic ring shown in Formula (I) a hetero ring system;
R² is one or more substituents independently selected from the group consisting of H, alkyl, alkenyl, alkynyl, aryl, alkoxy, aryloxy, halogen, haloalkyl, hydroxyalkyl and —NO₂, wherein each of said alkyl, alkenyl, alkynyl, aryl, alkoxy and aryloxy can be unsubstituted or optionally substituted with one or more moieties which can be the same or different, each moiety being independently selected from the group consisting of hydroxyl, halogen, alkyl, haloalkyl, aryl, haloaryl, alkylaryl, arylalkyl, cycloalkyl, aryloxy, alkoxy substituted with aryl, alkyl substituted with heterocyclyl, aryl substituted with haloaklkyl, aryl substituted with cycloalkyl, aryl substituted with arylalkyl, aryl substituted with alkoxy, aryl substituted with aryloxy, aryl substituted with —O-arylalkyl, aryl substituted with aryl, aryloxy, aryloxy substituted with alkyl, aryloxy substituted with cycloalkyl and aryloxy substituted with arylalkyl,
or optionally R² represents two substituents which are joined to form together with the aromatic ring shown in Formula I a substituted or unsubstituted ring or hetero ring system, or optionally at least one of R² is joined to the —NH—R³-group to form together with the aromatic ring shown in Formula (I) a hetero ring system;
R³ is —C(O)-alkyl, —C(O)O-alkyl, —C(O)NH-alkyl, —C(O)-cycloalkyl, —C(O)O-cycloalkyl, —C(O)NH-cycloalkyl, —C(O)-aryl, —C(O)O-aryl, —C(O)NH-aryl, —C(O)-heteroaryl, —C(O)O— heteroaryl, —C(O)NH-heteroaryl, aryl substituted with R⁵, heteroaryl substituted with R⁵, —S(O₂)—R⁹ or 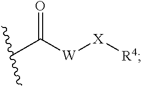
wherein
W is —(CH₂)ₙ—, —O—(CH₂)ₙ—, —NH—(CH₂)ₙ—, —(CH₂)ₚ-L-(CH₂)_{q}—,
—O—(CH₂)ₚ-L-(CH₂)_{q}— or —NH—(CH₂)ₚ-L-(CH₂)_{q}—,
n is an integer from 1 to 6;
p and q are integers independently selected from 0 to 6;
L is a linking group selected from the group consisting of heteroaryl, aryl, heterocyclyl and cycloalkyl;
X is O, S, NH, CH₂, SO or CO, wherein X preferably is O or S or NH or CH₂;
R⁴ is aryl or heteroaryl, wherein said aryl or heteroaryl can be unsubstituted or optionally substituted with one or more moieties which can be the same or different, each moiety being independently selected from the group consisting of H, halogen, alkyl, cycloalkyl, heterocyclyl, aryl, heteroaryl, alkoxy, aryloxy, arylalkyl, alkylaryl, haloaryl, haloalkylaryl, haloalkyl and trialkylsilyl;
R⁵ is aryloxy or arylalkyl or optionally R⁵ represents two substituents linked to each other to form together with the aryl or heteroaryl a polycyclic ring system; and
R⁹ is alkyl, cycloalkyl or —W—X—R⁴;
with the proviso that, when R³ is —C(O)-alkyl and the alkyl is methyl, then R² is not H, halogen, phenyl, biphenyl or 2-Cl-4-CF₃-phenoxy;
with the further proviso that, when R³ is —C(O)-alkyl and the alkyl is ethyl or cyclopropyl, then R² is not alkyl substituted with a phenyl ring which is unsubstituted at the 4-position, alkenyl substituted with a phenyl ring which is unsubstituted at the 4-position, alkynyl substituted with a phenyl ring which is unsubstituted at the 4-position, alkyoxy substituted with a phenyl ring which is unsubstituted at the 4-position, or aryloxy wherein the aryl of the aryloxy is a phenyl ring which is unsubstituted at the 4-position;
with the further proviso that, when R³ is —C(O)-alkyl and the alkyl is substituted with halogen, then R² is not alkyl or halogen;
with the further proviso that, when R³ is aryl substituted with R⁵ and R⁵ is aryloxy, then the aryloxy is not phenoxy
with the further proviso that, when R³ is —C(O)NH-aryl, then the aryl is not substituted with aryl or halogen.

14. Compound, or pharmaceutically acceptable salts or solvates of said compound, according to claim 13 having the following structure: 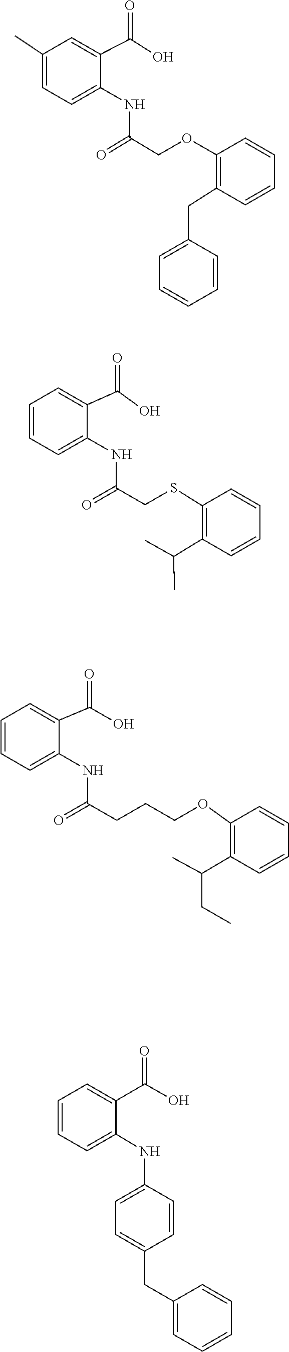 [SMILES: Cc1ccc(NC(=O)COc2ccccc2Cc2ccccc2)c(C(=O)O)c1 ; CC(C)c1ccccc1SCC(=O)Nc1ccccc1C(=O)O ; CCC(C)c1ccccc1OCCCC(=O)Nc1ccccc1C(=O)O ; O=C(O)c1ccccc1Nc1ccc(Cc2ccccc2)cc1] 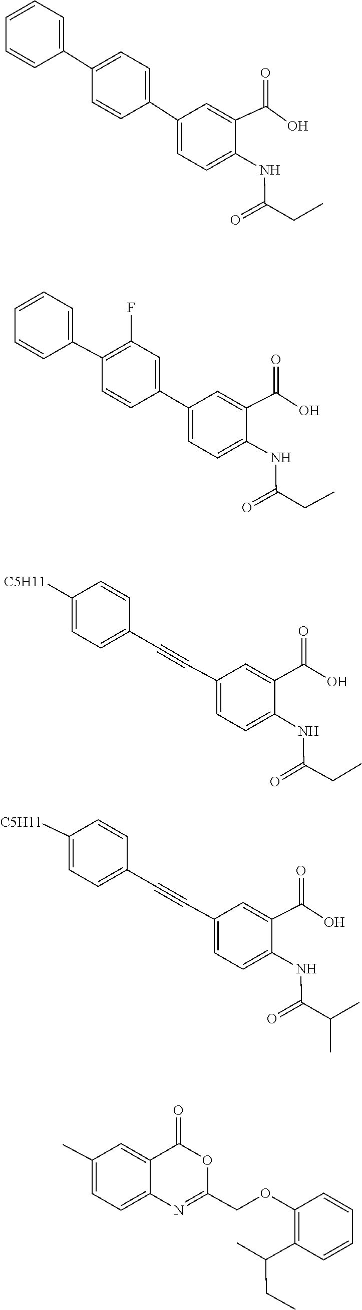 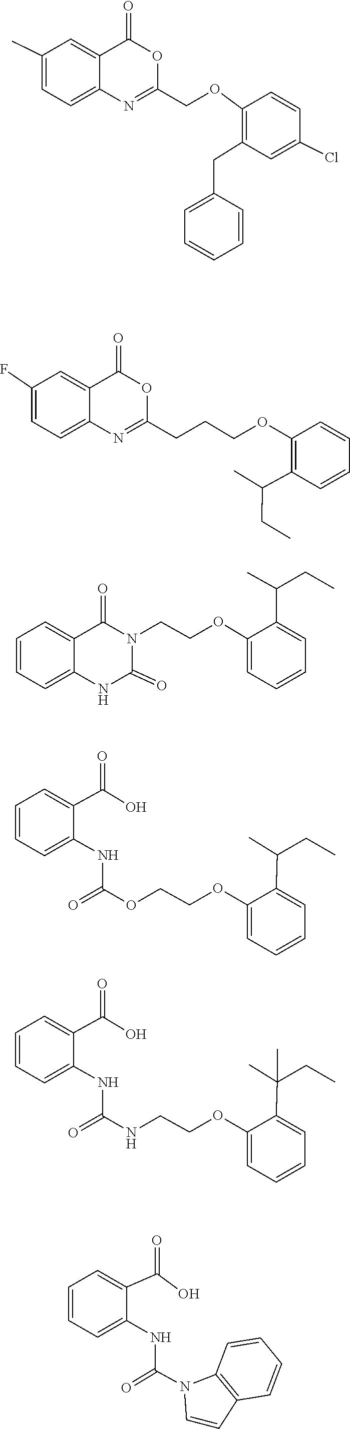 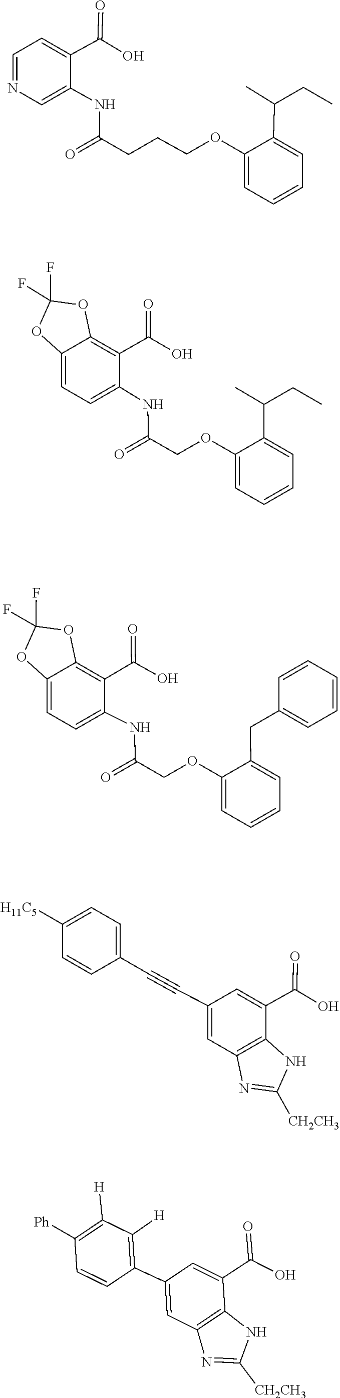 [chemical structure]
